(19) United States
(12) Patent Application Publication
(10) Pub. No.: US 2023/0227902 A1
(43) Pub. Date: Jul. 20, 2023

(54) METHOD OF REPEATEDLY MOVING A DOUBLE-STRANDED POLYNUCLEOTIDE THROUGH A NANOPORE

(71) Applicant: Oxford Nanopore Technologies PLC, Oxford (GB)

(72) Inventors: James Edward Graham, Oxford (GB); Christopher Peter Youd, Oxford (GB); Clive Gavin Brown, Oxford (GB); Marion Louise Crawford, Oxford (GB); Andrew John Heron, Oxford (GB); James White, Oxford (GB)

(73) Assignee: Oxford Nanopore Technologies PLC, Oxford (GB)

(21) Appl. No.: 18/010,824

(22) PCT Filed: Jun. 18, 2021
(86) PCT No.: PCT/GB2021/051557

§ 371 Date: Dec. 16, 2022

(30) Foreign Application Priority Data
Jun. 18, 2020 (GB) 2009349.8

(51) Int. Cl. C12Q 1/6869

(52) U.S. Cl. CPC C12Q 1/6869

(57) ABSTRACT

Provided herein is a method of moving a double-stranded polynucleotide with respect to a nanopore using a motor protein. The method allows a portion of the polynucleotide to be interrogated by the pore multiple times. Also provided are polynucleotide adapters and kits comprising such adapters. The methods find use in characterising polynucleotides, for example in sequencing.

### RELATED APPLICATIONS

This application is a national stage filing under 35 U.S.C. § 371 of international PCT application PCT/GB2021/051557, filed Jun. 18, 2021, which claims the benefit of Great Britain application number 2009349.8, filed Jun. 18, 2020, each of which is herein incorporated by reference in its entirety.

### Reference to a Sequence Listing Submitted as a Text File Via EFS-Web

This application contains a Sequence Listing which has been submitted in ASCII format via EFS-Web and is hereby incorporated by reference in its entirety. Said ASCII copy, created on Dec. 15, 2022, is named 0036670134US00-SEQ-KZM and is 46,237 bytes in size.

### FIELD

The present disclosure provides methods of moving polynucleotides with respect to detectors such as transmembrane nanopores. The disclosure also provides methods of characterising polynucleotides and methods of encoding data on polynucleotides using the disclosed methods of moving polynucleotides. Novel polynucleotide adapters and kits for use in such methods are also provided.

### BACKGROUND

Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore.

Polynucleotides are important analytes for sensing in this manner. Nanopore sensing of polynucleotide analytes can reveal the identity and perform single molecule counting of the sensed analytes, but can also provide information on their composition such as their nucleotide sequence, as well as the presence of characteristics such as base modifications, oxidation, reduction, decarboxylation, deamination and more. Nanopore sensing has the potential to allow rapid and cheap polynucleotide sequencing, providing single molecule sequence reads of polynucleotides of tens to tens of thousands bases length.

Two of the essential components of polymer characterization using nanopore sensing are (1) the control of polymer movement through the pore and (2) the discrimination of the component building blocks as the polymer is moved through the pore. During nanopore sensing of analytes such as polynucleotides, it is important to control the movement of the polynucleotide with respect to the pore. Uncontrolled movement can prevent or impede accurate characterisation of the polynucleotides. For example, accurately distinguishing each nucleotide in a homopolymeric polynucleotide is problematic when the movement of the polynucleotide with respect to the pore is not controlled.

There is further an ongoing need to store data efficiently and over prolonged time scales. However, the physical media used to store data today are prone to deterioration over time with associated loss of data fidelity. Accordingly, one approach that has been proposed is to use polynucleotides such as DNA to encode data for long-term data storage.

Most attempts to date have focused on de novo synthesis of DNA sequences which encode data. For example, binary data may be encoded in blocks of DNA. The DNA blocks may be synthesized in vitro by conventional means such as phosphoramidate chemistry. Such DNA blocks may include auxiliary information such as address sequences, amplification or sequencing tags, and the like. To encode large amounts of data, multiple blocks are used with the blocks being ordered according to their address sequences. However, such encoding steps are very slow and costly. In particular, such encoding steps may be limited by the synthesis of the DNA. Various approaches have been proposed for decoding data thus encoded. Some approaches have involved amplification of the DNA following by conventional sequencing technologies, such as Sanger sequencing. However, such approaches are not feasible for large-scale data storage and readout as throughput speed is limited; plus the amplification steps involved can introduce errors. Next-generation sequencing methods have been proposed but do not overcome the inherent limitations in the encoding/synthesis steps.

A method of encoding data on a polynucleotide strand by controlling the movement of the polynucleotide strand with respect to a nanoreactor such as a nanopore is provided in PCT/GB2019/053669, the entire contents of which are hereby incorporated by reference. The method disclosed in PCT/GB2019/053669 comprises controlling the movement of the polynucleotide with respect to the nanoreactor and selectively modifying portions of the polynucleotide strand as it moves with respect to the nanoreactor. The modifications to the strand can encode data on the strand. The methods disclosed in PCT/GB2019/053669 are extremely robust, are scalable, allow long write lengths, and allow high density storage of data.

In such methods, controlling the movement of the polynucleotide with respect to the nanoreactor (e.g. the nanopore) is important. Uncontrolled movement of the polynucleotide can lead to loss of fidelity in the data being written and/or loss of data read from the encoded polynucleotide.

Accordingly, both for characterising polynucleotides and for encoding data on polynucleotides there is a need for new methods of controlling the movement of polynucleotides with respect to a detector such as a nanopore.

It is known to use a motor protein to control the movement of a polymer such as a polynucleotide. Suitable motor proteins include polynucleotide-handling enzymes such as helicases, exonucleases, topoisomerases and the like. The motor protein processes the polynucleotide in a controlled manner. The motor protein can thus be used to control the movement of a polymer such as a polynucleotide with respect to a nanopore.

Whilst the use of motor proteins has allowed significant improvements to be achieved in the processing of polynucleotides e.g. for characterisation by nanopore sensing, technical challenges remain. One issue is that the re-processing of regions of interest on the polynucleotide is challenging. For example, in some scenarios it is desirable to re-characterise a particular region of interest in order to improve accuracy of the characterisation of that region. In other scenarios it is desirable to repeatedly pass a particular region of a polynucleotide through a nanoreactor such that the region of the polynucleotide can be efficiently modified, e.g. to encode data on the polynucleotide. Motor proteins are typically monodirectional; i.e. they process polynucleotides such as DNA in a 5′→3′ or 3′→5′ direction. Accordingly, such proteins typically cannot control the movement of a polynucleotide with respect to a detector such as a nanopore such that a specific region of the polynucleotide can be repeatedly re-processed.

Another issue is that the fuel required for motor proteins to operate typically needs to be included in the reaction medium in order for such proteins to function. The requirement to include fuel molecules such as ATP in the reaction medium increases costs and complexity. In addition, it effectively limits the timescale at which the motor proteins can be used, as once the fuel molecules have been consumed the motor protein will no longer function. The outcome of this is that typically the speed at which a polynucleotide can be processed under the control of a motor protein drops off over time, as the fuel molecules are consumed.

It is also known that the movement of a polynucleotide with respect to nanopore detectors can be controlled by application of a voltage. For example, typically a positive voltage applied across a nanopore can be used to capture a polynucleotide strand and move it through the nanopore, whereas a negative voltage can be used to eject the strand. Whilst controlling the voltage applied can alter the direction of movement of a polynucleotide with respect to a nanopore, the movement achieved is typically uncontrolled. Such methods do not allow for specific regions of the polynucleotide to be selectively re-processed.

Accordingly, there is a need for new and/or improved methods of moving polynucleotides with respect to detectors such as nanopores.

### SUMMARY

The disclosure relates to a method of moving a double-stranded polynucleotide with respect to a detector using a motor protein. More particularly, the disclosure relates to methods involving the interplay of competing forces: hybridisation forces between strands of a double-stranded polynucleotide; forces applied by a motor protein; and forces applied in the interaction of the polynucleotide with the detector. Whilst the disclosure provides nanopores as exemplary detectors, the methods provided herein are amenable to detectors including (i) a zero-mode waveguide, (ii) a field-effect transistor, optionally a nanowire field-effect transistor; (iii) an AFM tip; (iv) a nanotube, optionally a carbon nanotube and (v) a nanopore.

In some embodiments, the disclosure relates to a method of moving a double-stranded polynucleotide with respect to a nanopore using a motor protein. The method typically involves the controlled interplay between competing forces: hybridisation forces between the two strands of the double-stranded polynucleotide; forces applied by the motor protein which processes the polynucleotide; and forces applied relative to the detector (e.g. across the nanopore) e.g. by voltage potentials and/or by polynucleotide binding proteins which process the polynucleotide. The methods allow the movement of the polynucleotide back and forth with respect to the detector (e.g. nanopore) thus allowing a selected region of the polynucleotide to be selectively processed, e.g. to be characterised and/or to have data encoded thereon. The motor protein used in the methods is chosen or modified such that its active polynucleotide-unwinding activity is suppressed. In this way, the requirement to include fuel molecules in the reaction medium is reduced or avoided, and the associated speed drop off over time is likewise reduced.

Accordingly, provided herein is a method of moving a double-stranded polynucleotide with respect to a detector, comprising:

a) contacting the polynucleotide with a motor protein and a detector;

b) allowing the double-stranded polynucleotide to move in a first direction with respect to the detector under conditions such that (i) a first portion of the double-stranded polynucleotide dehybridises and (ii) the motor protein controls the movement of one strand of the first portion of the double-stranded polynucleotide in the first direction with respect to the detector;

c) allowing the double-stranded polynucleotide to move in a second direction with respect to the detector under conditions such that (i) the strand of the first portion of the double stranded polynucleotide moves in the second direction with respect to the detector and (ii) at least part of the first portion of the polynucleotide rehybridises; and

d) allowing the double-stranded polynucleotide to move in the first direction with respect to the detector under conditions such that (i) a second portion of the double-stranded polynucleotide dehybridises and (ii) the motor protein controls the movement of one strand of the second portion of the double-stranded polynucleotide in the first direction with respect to the detector;

wherein the active double stranded polynucleotide-unwinding activity of the motor protein is suppressed.

Typically the detector is a nanopore.

Accordingly, provided herein is a method of moving a double-stranded polynucleotide with respect to a nanopore, comprising:

a) contacting the polynucleotide with a motor protein and a nanopore;

b) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;

c) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises; and

d) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore;

wherein the active double stranded polynucleotide-unwinding activity of the motor protein is suppressed.

In some embodiments, the first portion of the double-stranded polynucleotide is the same as the second portion of the double-stranded polynucleotide. In some embodiments the first portion of the double-stranded polynucleotide partially overlaps with the second portion of the double-stranded polynucleotide.

In some embodiments the method further comprises:

e) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises.

In some embodiments, steps (d) and (e) are repeated multiple times. In some embodiments, at each repeat, the second portion of the double-stranded polynucleotide partially overlaps with the second portion of the double-stranded polynucleotide of the preceding repeat.

In some embodiments, allowing the double-stranded polynucleotide to move in the first direction with respect to the nanopore comprises applying a first force to the double-stranded polynucleotide. In some embodiments, the first force exceeds the rehybridisation force of the polynucleotide.

In some embodiments, allowing the double-stranded polynucleotide to move in the second direction with respect to the nanopore comprises applying a second force to the double-stranded polynucleotide. In some embodiments, the second force is applied in the same direction relative to the nanopore as the first force, and the second force is exceeded by the rehybridisation force of the polynucleotide.

In some embodiments, the first force and/or the second force comprises a voltage potential. In some embodiments, the first force comprises a voltage potential and the second force comprises a voltage potential, and the first force is greater than the second force.

In some embodiments, the second force is applied in the opposite direction relative to the nanopore as the first force. In some embodiments, the second force comprises a force applied by a polynucleotide-handling enzyme which moves the polynucleotide in the second direction relative to the nanopore.

In some embodiments, the first force comprises a voltage potential and the second force comprises (i) a voltage potential applied in the same direction relative to the nanopore as the first force; and (ii) a force applied by a polynucleotide-handling enzyme which moves the polynucleotide in the opposite direction relative to the nanopore as the first force; and the component of the second force applied by the polynucleotide-handling enzyme exceeds the component of the second force applied by the voltage potential.

In some embodiments, the polynucleotide-handling enzyme is a helicase or a variant thereof. Preferably the polynucleotide-handling enzyme comprises the sequence of SEQ ID NO: 7 or a variant thereof or the sequence of SEQ ID NO: 8 or a variant thereof.

In some embodiments the second force comprises a physical force applied by a controllable anchoring point for the polynucleotide. In some embodiments the anchoring point is an atomic force microscopy (AFM) tip.

In some embodiments, the first force comprises a voltage potential and the second force comprises (i) a voltage potential applied in the same direction relative to the nanopore as the first force; and (ii) a force applied by a controllable anchoring point (e.g. an AFM tip) for the polynucleotide which moves the polynucleotide in the opposite direction relative to the nanopore as the first force; and the component of the second force applied by the controllable anchoring point for the polynucleotide exceeds the component of the second force applied by the voltage potential.

In some embodiments, the movement of the polynucleotide in the first direction is faster than the movement of the polynucleotide in the second direction.

In some embodiments, the active double stranded polynucleotide-unwinding activity of the motor protein is suppressed by omitting fuel for the motor protein from the reaction medium. In some embodiments, the motor protein is a variant in which NTP binding and/or hydrolysis is abolished or suppressed. In some embodiments, the motor protein is a variant in which DNA-processing activity is abolished or suppressed. In some embodiments, the motor protein is a helicase variant in which the pin domain has been removed or reduced. In some embodiments, the motor protein is a helicase or a variant thereof. Preferably the motor protein comprises the sequence of SEQ ID NO: 6 or a variant thereof.

Also provided is a method of characterising a double-stranded polynucleotide analyte, comprising carrying out a method as described herein; wherein one or more of steps (b), (c), (d) and (e) if present comprise taking one or more measurements as the double stranded polynucleotide moves with respect to the nanopore, wherein the one or more measurements are indicative of one or more characteristics of the polynucleotide, and thereby characterising the polynucleotide as it moves with respect to the nanopore.

Also provided is a method of characterising a target double-stranded polynucleotide analyte, comprising:

a) contacting the polynucleotide with a motor protein and a nanopore;

b1) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;

b2) taking one or more or more measurements indicative of one or more characteristics of the target polynucleotide as the double stranded polynucleotide moves in the first direction with respect to the nanopore;

c1) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises;

c2) optionally taking one or more or more measurements indicative of one or more characteristics of the target polynucleotide as the double stranded polynucleotide moves in the second direction with respect to the nanopore;

d1) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore; and

d2) taking one or more or more measurements indicative of one or more characteristics of the target polynucleotide as the double stranded polynucleotide moves in the first direction with respect to the nanopore;

wherein the active double stranded polynucleotide-unwinding activity of the motor protein is suppressed.

In some embodiments, the method further comprises:

e1) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises; and

e2) optionally taking one or more or more measurements indicative of one or more characteristics of the target polynucleotide as the double stranded polynucleotide moves in the second direction with respect to the nanopore.

In some embodiments, the first and/or second portions; the movement of the polynucleotide; the motor protein and/or the polynucleotide-handling enzyme if present is as defined herein. In some embodiments the one or more measurements are one or more current measurements and/or one or more optical measurements.

Also provided is a method of encoding data on a double-stranded polynucleotide, comprising carrying out a method provided herein; wherein one or more of steps (b), (c), (d) and (e) if present comprise modifying the portion of the polynucleotide in the vicinity of the nanopore as the polynucleotide moves with respect to the nanopore.

Also provided is a method of encoding data on a double-stranded polynucleotide, comprising:

a) contacting the polynucleotide with a motor protein and a nanopore;

b1) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;

b2) modifying the portion of the polynucleotide in the vicinity of the nanopore as the double stranded polynucleotide moves in the first direction with respect to the nanopore;

c1) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises;

c2) optionally modifying the portion of the polynucleotide in the vicinity of the nanopore as the double stranded polynucleotide moves in the second direction with respect to the nanopore;

d1) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore; and

d2) modifying the portion of the polynucleotide in the vicinity of the nanopore as the double stranded polynucleotide moves in the first direction with respect to the nanopore;

wherein the active double stranded polynucleotide-unwinding activity of the motor protein is suppressed.

In some embodiments, the method further comprises:

e1) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises; and

e2) optionally modifying the portion of the polynucleotide in the vicinity of the nanopore as the double stranded polynucleotide moves in the second direction with respect to the nanopore.

In some embodiments, the first and/or second portions; the movement of the polynucleotide; the motor protein and/or the polynucleotide-handling enzyme if present is as defined herein. In some embodiments, modifying the polynucleotide comprises subjecting the portion of the polynucleotide in the vicinity of the nanopore to reaction conditions comprising (i) the presence, absence or concentration of one or more chemical reagent(s); (ii) the engagement of an enzyme with the polynucleotide strand under conditions that the enzyme modifies the nucleotides within the polynucleotide strand; (iii) the presence or absence of electromagnetic radiation; and/or (iv) the presence or absence of applied heat.

Also provided is a polynucleotide adapter having a motor protein and a polynucleotide-handling enzyme bound thereto, wherein the motor protein is capable of controlling the movement of the target polynucleotide with respect to a nanopore in a first direction; the polynucleotide-handling enzyme is capable of applying a force to move the target polynucleotide with respect to the nanopore in a second direction opposite to the first direction; and wherein the active double stranded polynucleotide-unwinding activity of the motor protein is suppressed.

Also provided is a kit for modifying a polynucleotide, comprising:

i) a polynucleotide adapter;

ii) a motor protein capable of controlling the movement of a target polynucleotide in a first direction with respect to a nanopore, wherein the active double stranded polynucleotide-unwinding activity of the motor protein is suppressed; and

iii) a polynucleotide-handling enzyme capable of applying a force to move the target polynucleotide in a second direction opposite to the first direction.

In some embodiments, the motor protein and polynucleotide-handling enzyme are as defined herein.

### BRIEF DESCRIPTION OF THE FIGURES

FIG. 1. Schematic diagram showing a non-limiting example of the movement of a polynucleotide in accordance with the methods provided herein; wherein the first portion of the double-stranded polynucleotide is the same as the second portion of the double-stranded polynucleotide.

FIG. 2. Schematic diagram showing a non-limiting example of the movement of a polynucleotide in accordance with the methods provided herein; wherein the first portion of the double-stranded polynucleotide overlaps with the second portion of the double-stranded polynucleotide.

FIG. 3. Schematic diagram showing a non-limiting example of the movement of a polynucleotide in accordance with the methods provided herein; wherein the first portion of the double-stranded polynucleotide does not overlap with the second portion of the double-stranded polynucleotide.

FIG. 4. Raw data showing examples of DNA translocating a nanopore under the control of a motor protein having suppressed polynucleotide-unwinding activity as defined herein. A: Raw data trace showing complete translocation of DNA fragment through a nanopore under the control of the motor protein. B: Data from (A), mapped to the genomic reference. Data are discussed in Example 1.

FIG. 5. Raw data showing examples of controlled DNA translocation back and forth through a nanopore under the control of a motor protein having suppressed polynucleotide-unwinding activity as defined herein. A: Schematic of the experimental setup, showing DNA moving through a nanopore embedded in a membrane under the control of the motor protein. The two opposing forces dictating the movement of the DNA, the force applied by the voltage, Fv, and the force applied by the rehybridization of the DNA, F_{H}, are represented by arrows. B: Raw data trace showing a DNA strand moving through a nanopore under the following voltage regimes: (a) A voltage of 180 mV is applied across the nanopore. The force applied by the voltage, Fv, is greater than the force of the DNA rehybridization, F_{H}, and the DNA translocates through the nanopore in the direction of Fv. (b) A voltage of 20 mV is applied across the nanopore. The force applied by the voltage, Fv, is less than the force of the DNA rehybridization, F_{H}, and the DNA translocates through the nanopore in the direction of F_{H}. C: Segments of raw data under voltage regime (a) were basecalled using a retrained RNN with Guppy software (v3.1.5, Oxford Nanopore), and the basecalls were aligned to the reference genome using minimap2 (https://github.com/lh3/minimap2, version 2.14-r883). The plot shows the reference locations of the basecall alignments on the y-axis, and the experiment time on the x-axis. Data are discussed in Example 2.

FIG. 6. Schematic diagram showing a non-limiting example of the movement of a polynucleotide in accordance with the methods provided herein. In this example movement of a double-stranded polynucleotide analyte is controlled by attaching the polynucleotide to an atomic force microscope (AFM) tip.

A double-stranded polynucleotide (A) is attached at one end, via one or both strands, to an AFM tip (B), for example via biotin-streptavidin interaction. A motor protein such as an inactivated helicase (C), loaded on an adapter, which bears a leader for capture, is attached to the other end. The motor protein may behaves as a ratchet to control movement of DNA through the nanopore. The leader is captured via an aperture (D), which may for example be a protein or solid-state nanopore embedded in a membrane (E). A voltage is applied across the membrane, which results in an electric force (Fe) on the DNA in the direction of the cis to the trans compartment. The AFM tip provides a counterbalancing force (Ft), which is initially lower in magnitude than the electric force or zero, and which may be used to further control the movement of the polynucleotide through the nanopore. The polynucleotide analyte is interrogated as follows: (i) the polynucleotide attached to the AFM tip is captured in the nanopore by applying a voltage in the direction from the cis to the trans compartment. (ii) the polynucleotide is engaged by the nanopore and the electric force begins to push on the motor protein. (iii) The motor protein moves along and passively unwinds the double-strand polynucleotide. The single strand of the polynucleotide on which the motor protein is loaded translocates in the direction of the electric force through the nanopore, whereas the displaced strand does not. (iv) At a desired time, the counterbalancing force provided by the AFM tip is increased so it exceeds the magnitude of the electric force; alternatively, the electric force is reduced by reducing the voltage. The relative reduction in the electrical force causes the AFM tip, polynucleotide analyte and motor protein to move out of the nanopore, and the displaced polynucleotide strand reanneals with the polynucleotide strand translocating through the nanopore, pushing the motor protein backwards to an earlier point on the strand (not necessarily the beginning). The forces are then reset to how they were in position (ii).

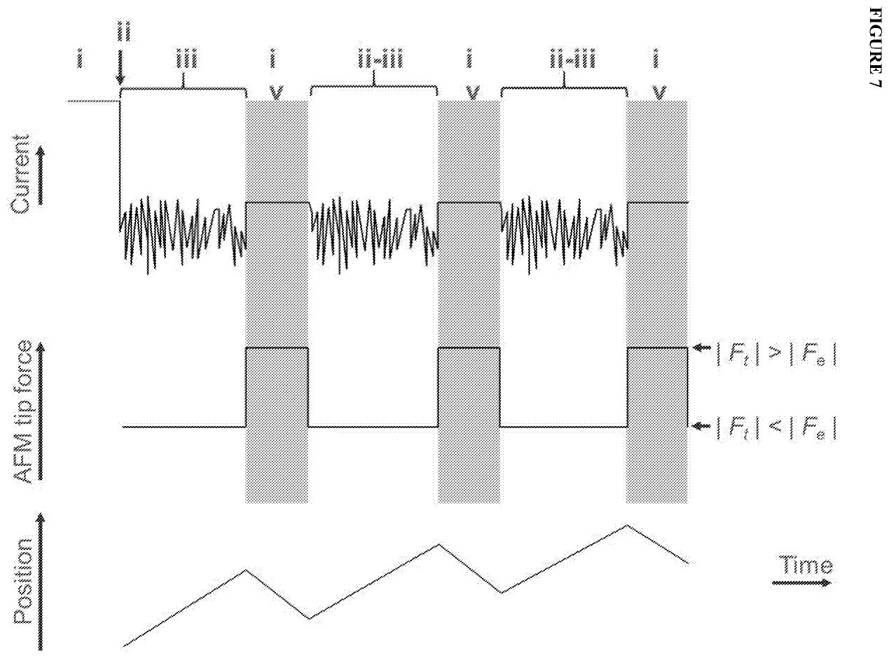

FIG. 7. Schematic diagram showing current traces for an AFM-driven unzipping/rezipping experiment for the experiment described in Example 3. Top panel: current-time trace for the experiment. Events (i)-(iv) correspond to the events described in FIG. 6: (i), open pore current; (ii), polynucleotide capture, (iii) motor protein-controlled translocation, (iv) reversal of direction and reannealing. Middle panel: force-time trace for the experiment, with AFM tip in constant force mode. Bottom panel: position of the motor protein on the sequenced strand. Grey boxes show where an increased force is periodically applied via the AFM tip.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a motor protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypepties that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, neutral |
| Asp | polar, hydrophilic, charged | Pro | hydrophobic, neutral |
| | (−) | | |
| Glu | polar, hydrophilic, charged | Gln | polar, hydrophilic, neutral |
| | (−) | | |
| Phe | aromatic, hydrophobic, | Arg | polar, hydrophilic, charged |
| | neutral | | (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Movement of Polynucleotides

The disclosure relates to methods of moving a double-stranded polynucleotide with respect to a detector such as a nanopore. As explained in more detail below, the methods allow the a portion of the polynucleotide to be repeatedly processed, e.g. in order to characterise and/or modify it. Polynucleotides suitable for use in the provided methods are described in more detail herein. Detectors such as nanopores suitable for use in the disclosed methods are also described in more detail herein.

The methods are based at least in part on the recognition that a double-stranded polynucleotide typically needs to be dehybridised in order to translocate a first strand of polynucleotide through or across a detector such as a nanopore e.g. from a first side to a second side. This is often because the internal dimensions of nanopores used in processing polynucleotides are too small to allow a double-stranded polynucleotide to traverse the nanopore without dehybridising, as the diameter of double-stranded polynucleotides is greater than the diameter of single-stranded polynucleotides.

Dehybridisation (“unzipping”) can be achieved in a multiplicity of ways, including by controlling the movement of the double-stranded polynucleotide through a pore using a motor protein. On the other hand, allowing the polynucleotide to move back through the pore provides a single stranded region which is amenable to re-hybridisiation. In other words, re-hybridisation provides a force which favours movement of the polynucleotide with respect to the nanopore in the opposite direction to that resulting from dehybridisation. By controlling these forces, the movement of the polynucleotide with respect to the nanopore can be controlled.

Dehybridisation may thus be achieved as a force is applied to move the double-stranded polynucleotide in a first direction with respect to a nanopore. The first direction may be for example from a first opening in the nanopore to a second opening in the nanopore. For example, the first direction may be in the direction of from the cis opening in a nanopore to the trans opening in the nanopore. The notation “cis” and “trans” openings in nanopores is routine in the art. For example, the cis opening of a nanopore typically faces the cis chamber of a nanopore device such as an apparatus as described herein having cis and trans chambers, and the trans opening typically faces the trans chamber.

However, when the force is applied as a voltage (for example), the dehybridisation is typically too fast and/or uneven for accurate processing of the polynucleotide as it moves with respect to the nanopore. There is thus a need to further control the movement of the polynucleotide as it moves in the first direction. In developing the presently claimed methods it has surprisingly been found that a motor protein in which the active double-stranded polynucleotide-unwinding activity has been suppressed can be beneficially used in this way, to control the movement of a double-stranded polynucleotide in a first direction with respect to a nanopore as the double-stranded polynucleotide dehybridises. The motor protein controls the movement of one strand of the polynucleotide with respect to the nanopore and in doing so the double-stranded polynucleotide dehybridises. Suitable motor proteins for use in the provided methods are described in more detail herein. The use of motor proteins in which the active double-stranded polynucleotide-unwinding activity has been suppressed can be beneficial in that the requirement for fuel molecules as discussed herein to be included in the reaction medium is reduced or eliminated. This can reduce or eliminate drop off in processing speed over time as the fuel molecules are consumed. Accordingly, in some embodiments of the methods provided herein processing speed drop-off is reduced or eliminated. In other words, processing speed consistency is improved.

The reverse movement of the polynucleotide with respect to the nanopore (i.e. in the second direction) can be achieved by allowing at least part of the previously dehybridised double-stranded polynucleotide to re-hybridise (“re-zipping”). By rehybridising the double-stranded polynucleotide, the single strand which threads the nanopore is effectively moved with respect to the nanopore in the opposite direction to the unzipping direction.

This can be further understood by way of a non-limiting illustrative example. Thus, in one embodiment, in a first step a double-stranded polynucleotide having a first strand and a second strand is contacted with a cis opening of a nanopore having a cis opening and a trans opening. The first strand of the double-stranded polynucleotide is moved under the control of a motor protein through the nanopore in the direction from the cis to the trans opening. This movement causes the double-stranded polynucleotide to dehybridise and the second strand is not passed through the nanopore; e.g the second strand may be dispersed into the medium contacting the cis opening of the nanopore. The movement in this first direction may for example be achieved by applying a force across the nanopore which promotes the movement of the polynucleotide in the cis-to-trans direction. For example, a positive voltage potential can be applied across the nanopore, with the positive potential attracting the negatively charged polynucleotide to the trans side of the nanopore and thus promoting its movement through the nanopore under the control of the motor protein.

In a second step, the polynucleotide may be then allowed to move in the reverse direction. The movement in the reverse direction causes the first strand of the double-stranded polynucleotide which has threaded the nanopore to move back through the nanopore in the reverse direction (e.g. in the trans-to-cis direction). As the first strand of the double-stranded polynucleotide moves back through the nanopore it rehybridises to the second strand of the double-stranded polynucleotide. The rehybridisation of the first strand to the second strand provides a driving force for the movement of the polynucleotide in the reverse second direction. In some embodiments the rehybridisation force can be countered or offset by an applied force across the nanopore in order to control the movement of the double-stranded polynucleotide with respect to the nanopore. For example, a positive voltage potential can be applied across the nanopore, with the positive potential attracting the negatively charged polynucleotide and thus countering the rehybridisation force. Other forces can be applied as described herein.

In a third step, the double-stranded polynucleotide may then be allowed to move back in the first direction, e.g. in the cis-to-trans direction again as previously described.

In the first step, a first portion of the double-stranded polynucleotide dehybridises. In the second step at least a part of that first portion rehybridises. In the third step a second portion of the polynucleotide dehybridises. As will be apparently, typically at least part of the second portion of the polynucleotide will be comprised in the first portion, i.e. was previously moved through the nanopore in the first step. Thus, that portion may be re-processed by the nanopore. When the nanopore is used to characterise the polynucleotide the portion that repeatedly passes through the nanopore may be repeatedly recharacterised. When the nanopore is used to encode data on the polynucleotide by modifying the polynucleotide, the portion of the polynucleotide that repeatedly passes through the nanopore may be repeatedly modified.

The movement may thus be considered as “flossing” the polynucleotide back and forward through the nanopore. In other words, the movement may thus be considered as moving the polynucleotide x steps through the nanopore in a first direction; y steps back through the nanopore in the second direction; and z steps through the nanopore in the first direction.

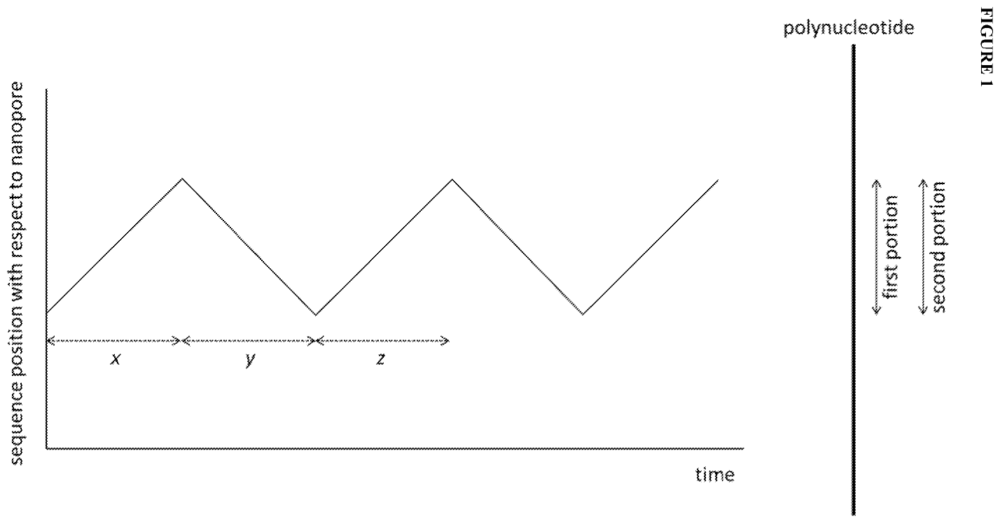

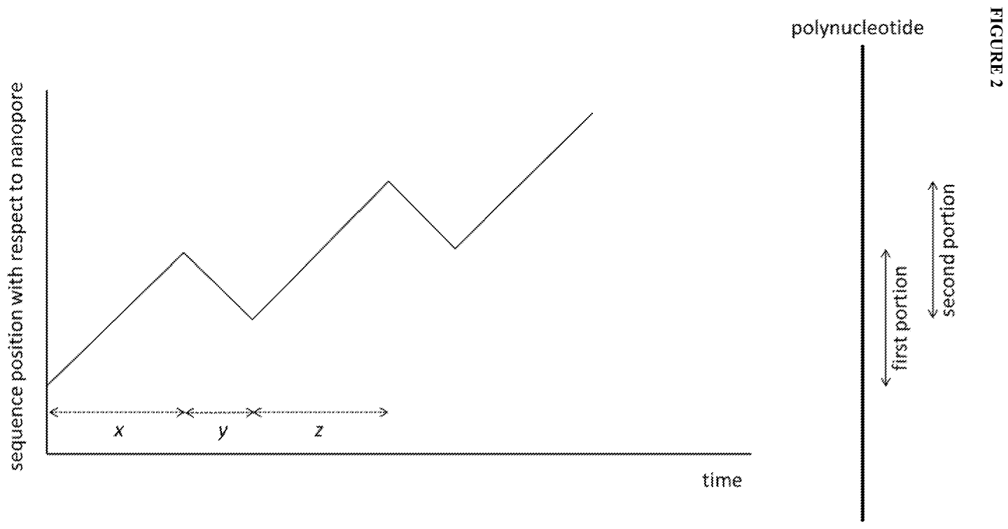

In some embodiments the first portion of the double-stranded polynucleotide is the same as the second portion of the double-stranded polynucleotide. In other words, the same portion of the polynucleotide may be flossed back and forwards through the nanopore. This may be achieved if, for example, x=y=z. This is shown schematically in FIG. 1.

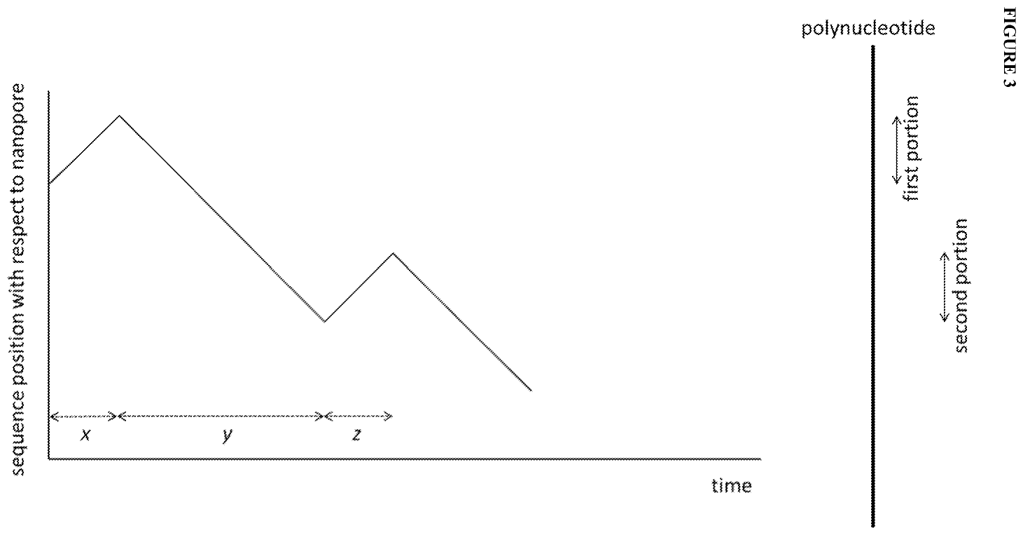

In some embodiments the first portion of the double-stranded polynucleotide partially overlaps with the second portion of the double-stranded polynucleotide. In other words, the polynucleotide may be ratcheted in a zig-zag manner through the pore. This may be achieved if, for example, x≠y. This is shown schematically in FIG. 2. In FIG. 2, x=z and x, z>y but those skilled in the art will appreciate that in other embodiments, x≠z (e.g. z>x or x>z); y>x, etc.

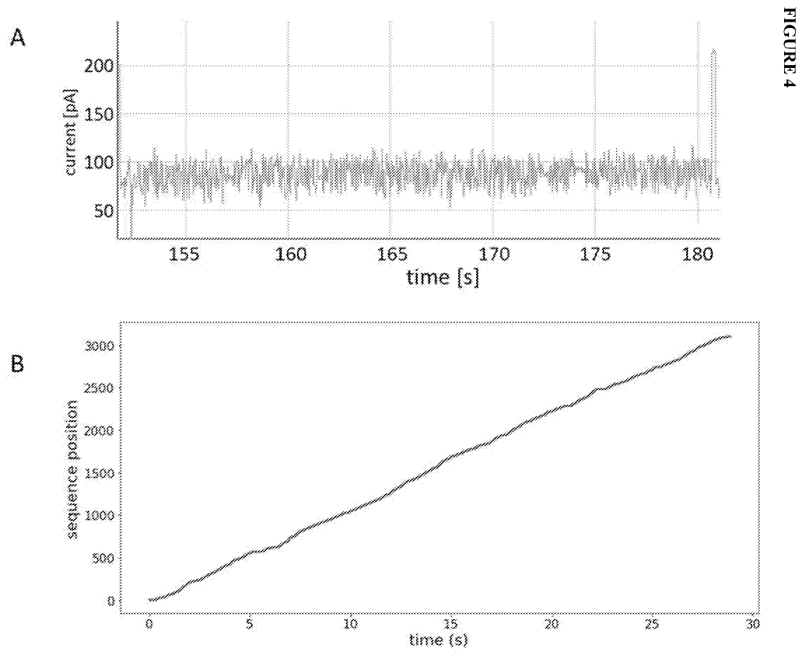

In some embodiments the first portion of the polynucleotide does not overlap with the second portion of the polynucleotide. This may be achieved if, for example, y>(x+z). This is shown schematically in FIG. 3.

Those skilled in the art will appreciate that in the methods provided herein, the portion of the polynucleotide that is processed in any of the steps of the disclosed methods is a parameter that can be controlled by the user of the method.

In some embodiments, the provided methods further comprise in addition to steps (a), (b), (c) and (d) described in more detail herein, the step of (e) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises. In other words, the disclosed methods may comprise moving the strand “forward” and “back” through the nanopore at least twice in each direction.

In some embodiments steps (d) and (e) are repeated multiple times. Repeating steps (d) and (e) of the methods corresponds to the repeated flossing of at least portions of the double-stranded polynucleotide through the nanopore. If steps (d) and (e) are repeated 1 time (and only 1 time) so that the method comprises steps (d) and (e) twice and only twice, the method will comprise steps (a), (b), (c), (d), (e), (d₁), and (e₁), and three portions of the polynucleotide will be moved with respect to the nanopore: a first portion in steps (b) and (c); a second portion in steps (d) and (e); and a third portion in steps (d₁) and (e₁). If steps (d) and (e) are repeated 2 times (and only 2 times) so that the method comprises steps (d) and (e) three times and only three times, the method will comprise steps (a), (b), (c), (d), (e), (d₁), (e₁), (d₂) and (e₂); and four portions of the polynucleotide will be moved with respect to the nanopore: a first portion in steps (b) and (c); a second portion in steps (d) and (e); a third portion in steps (d₁) and (e₁) and a fourth portion in steps (d₂) and (e₂). In other words, if steps (d) and (e) are repeated n times, then each repeat moves the (n+2)th portion of the polynucleotide with respect to the nanopore.

In embodiments of the methods provided herein in which steps (d) and (e) are repeated multiple times, steps (d) and (e) may be repeated at least once, such as at least 2 times, such as at least 3 times, e.g. at least 4 times, for example at least 5 times, e.g. at least 10 times, such as at least 20 times, for example at least 50 times, such as at least 100 times, e.g. at least 1000 times, such as at least 10,000 times, e.g. at least 100,000 times or more. By increasing the number of times that steps (d) and (e) are repeated, the processing of the double-stranded polynucleotide is increased. When the methods provided herein are used in the characterisation of the polynucleotide, repeating steps (d) and (e) multiple times can lead to improved characterisation, because the portion of the polynucleotide that is being interrogated by the nanopore is sampled multiple times, and thus any stochastic errors that may be recorded in the analysis become less statistically significant. The accuracy of the characterising data thus obtained can therefore be improved.

When the methods provided herein are used in the modification of the polynucleotide, repeating steps (d) and (e) multiple times can lead to improved modification, because the portion of the polynucleotide that is being interrogated by the nanopore is in contact with the modification conditions multiple times, and thus any potential incomplete modification that may occur on the first pass through the conditions can be remedied on subsequent passes through the conditions. By way of non-limiting example, this can be useful in embodiments where the modification conditions can only be briefly applied e.g. to prevent damage to the polynucleotide backbone. The extent of modification of the portion of the polynucleotide which is being thus modified can therefore be improved.

In some embodiments when steps (d) and (e) of the provided methods are repeated multiple times, at each repeat, the second portion of the double-stranded polynucleotide partially overlaps with the second portion of the double-stranded polynucleotide of the preceding repeat. When at each repeat the second portion of the double-stranded polynucleotide partially but not wholly overlaps with the second portion of the double-stranded polynucleotide of the preceding repeat the polynucleotide is ratcheted in a zig-zag manner through the pore, as shown schematically in FIG. 2. When at each repeat the second portion of the double-stranded polynucleotide wholly overlaps with the second portion of the double-stranded polynucleotide of the preceding repeat the same portion of the polynucleotide is flossed back and forwards through the pore, as shown schematically in FIG. 1.

### Applied Forces During Movement

In some embodiments of the disclosed methods, forces are applied to the polynucleotide during the methods. As explained above, the applied forces can be controlled in order to control the methods.

In some embodiments, allowing the double-stranded polynucleotide to move in the first direction with respect to the detector such as a nanopore comprises applying a first force to the double-stranded polynucleotide. In some embodiments the first force exceeds the rehybridisation force of the polynucleotide.

By controlling the first force, the movement of the polynucleotide in the first direction with respect to the nanopore can be controlled. For example, by increasing the first force the movement of the polynucleotide through the nanopore can be increased, e.g. the rate at which the polynucleotide moves through the pore can be increased.

The first force can be applied to counteract or exceed the rehybridisation force of the dehybridised polynucleotide. In other words, the methods provided herein comprise unzipping a double-stranded polynucleotide as it moves through the pore in a first direction. The movement of the polynucleotide in the first direction is counteracted by a rehybridisation force which promotes movement in the second direction. If the force applied to the polynucleotide exceeds the rehybridisation force then double-stranded polynucleotide will be dehybridised and the overall direction of movement of the polynucleotide will be through the pore in the first direction.

In some embodiments, allowing the double-stranded polynucleotide to move in the second direction with respect to the nanopore comprises applying a second force to the double-stranded polynucleotide.

In some embodiments, the second force is applied in the same direction relative to the nanopore as the first force, and the second force is exceeded by the rehybridisation force of the polynucleotide. Because in such embodiments the second force is exceeded by the rehybridisation force, the overall direction of the movement of the polynucleotide is through the pore in the second direction and the dehybridised first and second strands of the double-stranded polynucleotide are hybridised together.

In some embodiments the second force is applied in the opposite direction relative to the nanopore as the first force. In such embodiments the second force supplements the rehybridisation force. The combined rehybridisation force and the second force results in the polynucleotide moving in the second direction with respect to the nanopore and so the dehybridised first and second strands of the double-stranded polynucleotide are hybridised together.

In the methods provided herein, any suitable force can be applied. In some embodiments, no external force is applied across the nanopore. For example, in some embodiments no electrical potential is applied. Such embodiments are in some embodiments particularly suited to methods in which optical measurements are taken as the polynucleotide moves with respect to the nanopore.

In some embodiments, the first force and/or the second force comprises a voltage potential applied across the nanopore. The voltage potential may be applied using any suitable apparatus, such as an apparatus described herein. Suitable voltage potentials are described in more detail herein.

In some embodiments, therefore, the first force comprises a voltage potential and the second force comprises a voltage potential, and the first force is greater than the second force. In such embodiments, the first force and the second force are typically applied in the same direction relative to the nanopore, e.g. a positive voltage may be applied to the trans side of the nanopore as the first force in order to promote the dehybridisation of the double-stranded polynucleotide as the polynucleotide moves in the first direction; and a lower positive voltage may be applied to the trans side of the nanopore as the second force in order to allow the movement of the polynucleotide in the second direction so that the two strands of the double-stranded polynucleotide rehybridise.

In some embodiments the first force comprises a voltage potential and the second force comprises a voltage potential, and the second force is applied in the opposite direction to the first force. For example, a positive voltage may be applied to the trans side of the nanopore as the first force in order to promote the dehybridisation of the double-stranded polynucleotide as the polynucleotide moves in the first direction; and a negative voltage may be applied to the trans side of the nanopore as the second force in order to promote the movement of the polynucleotide in the second direction so that the two strands of the double-stranded polynucleotide rehybridise.

In some embodiments the first force comprises a voltage potential and no second force is applied. For example, a positive voltage may be applied to the trans side of the nanopore as the first force in order to promote the dehybridisation of the double-stranded polynucleotide as the polynucleotide moves in the first direction; and no voltage may be applied as the second force. In such embodiments the rehybridisation force is solely responsible for the movement of the polynucleotide in the second direction as the two strands of the double-stranded polynucleotide rehybridise.

In some embodiments the second force comprises a force applied by a polynucleotide-handling enzyme which moves the polynucleotide in the second direction relative to the nanopore. The force applied by a polynucleotide-handling enzyme may be applied in addition to a force applied e.g. as a voltage. The force applied by a polynucleotide-handling enzyme may be applied instead of an applied voltage. Suitable polynucleotide-handling enzymes are described in more detail herein.

When the second force comprises a force applied by a polynucleotide-handling enzyme, the polynucleotide-handling enzyme may be present on either the same side of the nanopore as the motor protein or the different side of the nanopore to the motor protein. For example, in some embodiments the motor protein is present on the cis side of the nanopore and controls the movement of the polynucleotide in the cis-to-trans direction; and the polynucleotide-handling enzyme is also present on the cis side of the nanopore and controls the movements of the polynucleotide in the trans-to-cis direction. In other embodiments the motor protein is present on the cis side of the nanopore and controls the movement of the polynucleotide in the cis-to-trans direction; and the polynucleotide-handling enzyme is present on the trans side of the nanopore and controls the movements of the polynucleotide in the trans-to-cis direction. Of course the reverse setup is also possible. Accordingly, in some embodiments the motor protein is present on the trans side of the nanopore and controls the movement of the polynucleotide in the trans-to-cis direction; and the polynucleotide-handling enzyme is also present on the trans side of the nanopore and controls the movements of the polynucleotide in the cis-to-trans direction. In other embodiments the motor protein is present on the trans side of the nanopore and controls the movement of the polynucleotide in the trans-to-cis direction; and the polynucleotide-handling enzyme is present on the cis side of the nanopore and controls the movements of the polynucleotide in the cis-to-trans direction.

In some embodiments, the first force comprises a voltage potential and the second force comprises (i) a voltage potential applied in the same direction relative to the nanopore as the first force; and (ii) a force applied by a polynucleotide-handling enzyme which moves the polynucleotide in the opposite direction relative to the nanopore as the first force; and the component of the second force applied by the polynucleotide-handling enzyme exceeds the component of the second force applied by the voltage potential. In these embodiments, the applied voltage potential can generate an ionic flow which can be used to characterise the polynucleotide as it moves with respect to the nanopore. The overall movement of the polynucleotide in the second direction however is determined by the polynucleotide-handling enzyme and the rehybridisation force.

In some embodiments the second force comprises a physical force applied by a controllable anchoring point for the polynucleotide.

In some embodiments the anchoring point is an atomic force microscopy (AFM) tip. In some embodiments the anchoring point is made of silicon, borosilicate or silicon nitride. In some embodiments the anchoring point is coated with a coating for facilitating binding of the polynucleotide. In some embodiments the coating comprises gold. In some embodiments Au-thiol bonds are used to bind the polynucleotide or a group for binding to the polynucleotide to the anchoring point. In some embodiments the anchoring point is coated with a chemical group for attaching the polynucleotide to the anchoring point.

In some embodiments the polynucleotide is attached to the anchoring point such as the AFM tip by a chemical linker. In some embodiments the polynucleotide is attached to the anchoring point by a covalent linker. In some embodiments the polynucleotide is attached to the anchoring point by a non-covalent linker. In some embodiments the polynucleotide is attached to the anchoring point by hybridising to a polynucleotide on the anchoring point. In some embodiments the polynucleotide is attached to the anchoring point by a polymeric linker. In some embodiments the polynucleotide is attached to the anchoring point using strepatavidin-biotin chemistry. In some embodiments the polynucleotide is attached to the anchoring point using chemistry described herein under the heading “Tags” (see below).

In some embodiments the anchoring point is biotinylated for reacting with a streptavidin-modified polynucleotide. In some embodiments the anchoring point is biotinylated by silanisation of the anchoring point with an appropriate silane e.g. with (3-aminopropyl)triethoxysilane, followed by reaction with NHS-PEG-biotin.

The force applied by a controllable anchoring point (e.g. an AFM tip) may be applied in addition to a force applied e.g. as a voltage. The force applied by a controllable anchoring point (e.g. an AFM tip) may be applied instead of an applied voltage.

When the second force comprises a force applied by a controllable anchoring point (e.g. an AFM tip), the controllable anchoring point (e.g. AFM tip) may be present on either the same side of the nanopore as the motor protein or the different side of the nanopore to the motor protein. For example, in some embodiments the motor protein is present on the cis side of the nanopore and controls the movement of the polynucleotide in the cis-to-trans direction; and the controllable anchoring point (e.g. AFM tip) is also present on the cis side of the nanopore and controls the movements of the polynucleotide in the trans-to-cis direction. In other embodiments the motor protein is present on the cis side of the nanopore and controls the movement of the polynucleotide in the cis-to-trans direction; and the controllable anchoring point (e.g. AFM tip) is present on the trans side of the nanopore and controls the movements of the polynucleotide in the trans-to-cis direction. Of course the reverse setup is also possible. Accordingly, in some embodiments the motor protein is present on the trans side of the nanopore and controls the movement of the polynucleotide in the trans-to-cis direction; and the controllable anchoring point (e.g. AFM tip) is also present on the trans side of the nanopore and controls the movements of the polynucleotide in the cis-to-trans direction. In other embodiments the motor protein is present on the trans side of the nanopore and controls the movement of the polynucleotide in the trans-to-cis direction; and the controllable anchoring point (e.g. AFM tip) is present on the cis side of the nanopore and controls the movements of the polynucleotide in the cis-to-trans direction.

In some embodiments, the first force comprises a voltage potential and the second force comprises a force applied by the controllable anchoring point (e.g. an AFM tip) in the opposite direction relative to the nanopore as the first force. In some embodiments allowing the double-stranded polynucleotide to move in the first direction comprises applying a lower force from the controllable anchoring point than that applied first force. In some embodiments allowing the double-stranded polynucleotide to move in the second direction comprises applying a higher force from the controllable anchoring point than that applied first force.

In some embodiments, the first force comprises a voltage potential and the second force comprises (i) a voltage potential applied in the same direction relative to the nanopore as the first force; and (ii) a force applied by a controllable anchoring point (e.g. an AFM tip) for the polynucleotide which moves the polynucleotide in the opposite direction relative to the nanopore as the first force; and the component of the second force applied by the controllable anchoring point for the polynucleotide exceeds the component of the second force applied by the voltage potential.

When the anchoring point is an AFM tip the AFM tip is typically operated in constant force mode e.g. at a force of from about 1 to about 100 pN e.g from about 5 to about 50 pN such as from about 10 to about 20 or about 30 pN.

When used to control the movement of the polynucleotide in the second direction against a voltage potential applied in the first direct, the force applied by the controllable anchoring point (e.g. the AFM tip) is typically at least about 1%, such as at least about 5%, e.g. at least about 10% higher than the force arising from the voltage potential. The force applied by the controllable anchoring point (e.g. the AFM tip) is typically at most about 20%, such as at most about 15%, e.g. at most about 10% higher than the force arising from the voltage potential. For example, when the first force on the polynucleotide is a voltage force of e.g. about 20 pN (which may for example arise from application of a voltage of about +180 mV), the second force applied by the controllable anchoring point is typically about 10% higher (e.g. about 22 pN).

Typically, in the methods provided herein, the movement of the polynucleotide in the first direction is faster than the movement of the polynucleotide in the second direction. This is a parameter that can be controlled by the user of the method; for example by controlling the magnitude and direction of the first and second forces applied across the nanopore. However, the movement in the first direction is typically faster than that in the second direction and so the polynucleotide can be characterised or modified efficiently as it moves in the first direction, and/or additional data can be obtained as the polynucleotide moves in the second direction. In order to speed up the movement in the first direction the magnitude of the first force (when applied in the same direction as the first movements) can be increased. In order to speed up movement in the second direction the magnitude of the second force can be decreased (when applied in the opposite direction to the second movement) or can be increased (when applied in the same direction as the second movement).

### Motor Protein

As those skilled in the art will appreciate, any suitable motor protein can be used in the methods and products provided herein.

The motor protein may be any protein that is capable of binding to a polynucleotide and controlling its movement with respect to a nanopore, e.g. through the pore. However, in the provided methods the active double stranded polynucleotide unwinding activity of the motor protein is suppressed.

In more detail, motor proteins such as helicases can typically control the movement of DNA in at least two active modes of operation (when is provided with all the necessary components to facilitate movement e.g. ATP and Mg²⁺) and one inactive mode of operation (when not provided with the necessary components to facilitate movement; or when the motor protein is modified in order to prevent the active mode).

When provided with all the necessary components to facilitate movement, a motor protein may move along a polynucleotide such as DNA in either a 5′-3′ direction or a 3′-5′ direction. Many motor proteins process polynucleotides such as DNA in a 5′-3′ direction. Motor proteins can be used to control the movement of the polynucleotide through the pore in an active manner as has been described previously.

However, when a motor protein is not provided with the necessary components to facilitate movement, or is modified in order to prevent it from actively controlling the movement of the polynucleotide with respect to the nanopore, it can still passively control the movement of the polynucleotide with respect to the nanopore. For example, the motor protein can bind to the polynucleotide and act as a brake slowing the movement of the polynucleotide when it is pulled into the pore by an applied field (e.g. by the first force in the methods provided herein). In the “inactive” mode it typically does not matter whether the DNA is captured either 3′ or 5′ down (i.e. moves through the nanopore in a 5′-3′ direction or in a 3′-5′ direction), as the applied force provides the impetus to move the polynucleotide through the nanopore. However, in such embodiments the motor protein may still control the movement of the polynucleotide with respect to the nanopore e.g. by acting as a brake. When in the inactive mode the movement control of a polynucleotide by a motor protein can be described in a number of ways including ratcheting, sliding and braking.

Accordingly, in the methods provided herein, the control of the movement of the polynucleotide in the first direction is typically achieved by the motor protein acting in a passive mode.

In the methods provided herein the active polynucleotide-unwinding activity of the motor protein is suppressed. The active polynucleotide unwinding activity can be suppressed in any suitable way. For example, in some embodiments the active double stranded polynucleotide-unwinding activity of the motor protein is suppressed by omitting fuel for the motor protein from the reaction medium. The fuel may be omitted from the reaction medium on both sides of the nanopore (e.g. in both the cis and trans chambers of the apparatus) or may be omitted only from the chamber containing the motor protein. For example, the fuel may be omitted only from the cis chamber in embodiments wherein the motor protein is present in the cis chamber and the first direction in the methods provided herein is the movement of the polynucleotide from cis to trans.

In some embodiments the motor protein is a variant in which NTP binding and/or hydrolysis is abolished or suppressed. For example, NTP binding and/or hydrolysis can be abolished or suppressed by introducing mutations into the NTP binding site in the motor protein. The binding site in the motor protein can typically be determined by structural analysis, e.g. by X-ray crystallography in the presence of the NPT; and/or by restriction analysis to determine the functional portion of the protein. In some embodiments one or more key residues in the NTP catalytic cycle can be removed e.g. by site-directed mutagenesis, e.g. using techniques as set out in Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

In some embodiments the motor protein is a variant in which DNA-processing activity is abolished or suppressed. For example, DNA processing activity can be abolished or suppressed by introducing mutations into the polynucleotide binding site in the motor protein. The polynucleotide binding site in the motor protein can typically be determined by structural analysis, e.g. by X-ray crystallography in the presence of the NPT; and/or by restriction analysis to determine the functional portion of the protein. In some embodiments one or more key residues in the polynucleotide-processing catalytic cycle can be removed e.g. by site-directed mutagenesis, e.g. using techniques as set out in Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

In some embodiments the motor protein is a helicase variant. Helicase variants are described in more detailed herein. In some embodiments the motor protein is a is a helicase variant in which the pin domain has been removed or reduced. This is described in more detail herein.

As explained above, some embodiments of the methods provided herein also comprise the use of a polynucleotide-handling enzyme, e.g. to contribute to a second force for moving the polynucleotide in a second direction relative to the nanopore such that the two strands of the polynucleotide re-hybridise. A polynucleotide-handling enzyme may be a motor protein as described herein, except that a polynucleotide-handling enzyme is not typically modified in order to suppress any active double stranded polynucleotide unwinding activity.

A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. A motor protein as used herein may be, or may be derived from a polynucleotide handling enzyme.

In one embodiment, the motor protein and/or polynucleotide-handling enzyme is derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

Typically, the motor protein and/or polynucleotide-handling enzyme is a helicase, a polymerase, an exonuclease, a topoisomerase, or a variant thereof.

In some embodiments, the motor protein and/or polynucleotide-handling enzyme may be modified to prevent the motor protein disengaging from the polynucleotide. The motor protein and/or polynucleotide-handling enzyme can be adapted in any suitable way. For example, the motor protein and/or polynucleotide-handling enzyme can be loaded on the polynucleotide and then modified in order to prevent it from disengaging from the polynucleotide. Alternatively, the motor protein and/or polynucleotide-handling enzyme can be modified to prevent it from disengaging from the polynucleotide before it is loaded onto the polynucleotide. Modification of a motor protein and/or a polynucleotide-handling enzyme in order to prevent it from disengaging from a polynucleotide can be achieved using methods known in the art, such as those discussed in WO 2014/013260, which is hereby incorporated by reference in its entirety, and with particular reference to passages describing the modification of motor proteins such as helicases in order to prevent them from disengaging with polynucleotide strands. For example, a motor protein and/or polynucleotide-handling enzyme can be modified by treating with tetramethylazodicarboxamide (TMAD).

For example, a motor protein and/or a polynucleotide-handling enzyme may have a polynucleotide-unbinding opening; e.g. a cavity, cleft or void through which a polynucleotide strand may pass when the motor protein/polynucleotide-handling enzyme disengages from the strand. In some embodiments, the polynucleotide-unbinding opening is the opening through which a polynucleotide may pass when the motor protein/polynucleotide-handling enzyme disengages from the polynucleotide. In some embodiments, the polynucleotide-unbinding opening for a given motor protein/polynucleotide-handling enzyme can be determined by reference to its structure, e.g. by reference to its X-ray crystal structure. The X-ray crystal structure may be obtained in the presence and/or the absence of a polynucleotide substrate. In some embodiments, the location of a polynucleotide-unbinding opening in a given motor protein/polynucleotide-handling enzyme may be deduced or confirmed by molecular modelling using standard packages known in the art. In some embodiments, the polynucleotide-unbinding opening may be transiently produced by movement of one or more parts e.g. one or more domains of the motor protein.

The motor protein/polynucleotide-handling enzyme may be modified by closing the polynucleotide-unbinding opening. Closing the polynucleotide-unbinding opening may therefore prevent the motor protein/polynucleotide-handling enzyme from disengaging from the polynucleotide. For example, the motor protein and/or polynucleotide-handling enzyme may be modified by covalently closing the polynucleotide-unbinding opening. In some embodiments, a preferred protein for addressing in this way is a helicase.

In one embodiment, the motor protein and/or the polynucleotide-handling enzyme is or is derived from an exonuclease. Suitable enzymes include, but are not limited to, exonuclease I from E. coli (SEQ ID NO: 1), exonuclease III enzyme from E. coli (SEQ ID NO: 2), RecJ from T. thermophilus (SEQ ID NO: 3) and bacteriophage lambda exonuclease (SEQ ID NO: 4), TatD exonuclease and variants thereof. Three subunits comprising the sequence shown in SEQ ID NO: 3 or a variant thereof interact to form a trimer exonuclease.

In one embodiment, the motor protein is and/or the polynucleotide-handling enzyme a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase (SEQ ID NO: 5) or a variant thereof. Modified versions of Phi29 polymerase that may be used in the invention are disclosed in U.S. Pat. No. 5,576,204.

In one embodiment the motor protein and/or the polynucleotide-handling enzyme is a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

In one embodiment, the motor protein and/or the polynucleotide-handling enzyme is a helicase. Any suitable helicase can be used in accordance with the methods provided herein. For example, the or each enzyme used in accordance with the present disclosure may be independently selected from a Hel308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep, PcrA, Pif1 and TraI. These helicases typically work on single stranded DNA. Examples of helicases that can move along both strands of a double stranded DNA include FtfK and hexameric enzyme complexes, or multisubunit complexes such as RecBCD.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

In one embodiment a helicase may comprise the sequence shown in SEQ ID NO: 6 (Trwc Cba) or a variant thereof, the sequence shown in SEQ ID NO: 7 (Hel308 Mbu) or a variant thereof or the sequence shown in SEQ ID NO: 8 (Dda) or a variant thereof. Variants may differ from the native sequences in any of the ways discussed herein. An example variant of SEQ ID NO: 8 comprises E94C/A360C. A further example variant of SEQ ID NO: 8 comprises E94C/A360C and then (ΔM1)G1G2 (i.e. deletion of M1 and then addition of G1 and G2).

As mentioned above, in some embodiments of the methods provided herein, the motor protein is a helicase variant in which the pin domain has been removed or reduced.

Although structural variation within helicases is known, certain features are common to many helicases. For example, helicases typically comprise structural elements known as “pins” or “wedges” which are believed to act as physical barriers which help to separate strands of double-stranded polynucleotides.

Many helicases comprise pins comprised of β-hairpin structures, often comprising aromatic residues that base stack with duplex DNA at the ss/dsDNA junction. Wedges may also comprise larger domains that act as physical unwinding elements in helicases. The location and sequence of such features can be determined by those of skill in the art, for example via crystallographic studies of such proteins in presence of DNA and fuel molecules such as ATP. The location and sequence of relevant pins and wedges for exemplary helicases are known in the art and are discussed in, for example, DNA Helicases and DNA Motor Proteins, Spies (ed), Springer. In the methods provided herein, the motor protein can comprise modifications in a pin and/or wedge domain in order to eliminate or suppress DNA-unwinding activity. The pin/wedge domain can for example be removed by deletion mutations.

Other modifications which can be made to a motor protein in order to suppress DNA-unwinding activity include modification of the cofactor (e.g. Mg²⁺) binding domain.

In one embodiment the motor protein comprises the sequence shown in SEQ ID NO: 6 (Trwc Cba) or a variant thereof. Trwc Cba is also known as TraI Cba. In some embodiments the variant of SEQ ID NO: 6 comprises (or only comprises) (a) Q594A, (b) L376C/Q594A/K762C, (c) L376C/Q594A/A779C, (d) Q346C/Q594A/A779C, (e) Q346C/Q594A/A783C, (f) D411/Q594A/A783C, (g) Q594A/R353C/E722C, (h) Q594A/Q357C/T720C, (i) Q594A/R358C/T720C, (j) Q594A/H354C/T720C, (k) Q594A/F374C/E722C or (1) Q594A/S350C/E722C. Any of (a) to (1) may further comprise and then (ΔM1)G1G2 (i.e. deletion of M1 and then addition of G1 and G2). In one embodiment the motor protein comprises the sequence shown in SEQ ID NO: 9 (TraI Cba L376C/Q594A/K762C). SEQ ID NO: 9 is a variant of SEQ ID NO: 6.

In some embodiments the motor protein comprises the sequence shown in SEQ ID NO: 6 (Trwc Cba) or a variant thereof and the polynucleotide-handling enzyme if present comprises the sequence shown in SEQ ID NO: 7 (Hel308 Mbu) or a variant thereof or the sequence shown in SEQ ID NO: 8 (Dda) or a variant thereof.

Typically, a motor protein or polynucleotide-handling enzyme may have a fuel binding site. The active unwinding of DNA may be coupled to fuel hydrolysis, e.g. in the polynucleotide-handling enzyme. As explained above in more detail, the methods provided herein use a motor protein in which the DNA-unwinding activity is suppressed and this can reduce fuel turnover.

Fuel is typically free nucleotides or free nucleotide analogues. The free nucleotides may be one or more of, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are usually selected from AMP, TMP, GMP, CMP, UMP, dAMP, dTMP, dGMP or dCMP. The free nucleotides are typically adenosine triphosphate (ATP).

A cofactor for the motor protein is a factor that allows the motor protein to function. The cofactor is preferably a divalent metal cation. The divalent metal cation is preferably Mg²⁺, Mn²⁺, Ca²⁺ or Co²⁺. The cofactor is most preferably Mg²⁺.

### Polynucleotide

The methods of the invention involve moving a double-stranded polynucleotide with respect to a detector such as a nanopore.

A polynucleotide, such as a nucleic acid, is a macromolecule comprising two or more nucleotides. A polynucleotide can be single-stranded or double-stranded. A double-stranded polynucleotide is made of two single stranded polynucleotides hybridised together.

A polynucleotide may comprise any combination of any nucleotides. The nucleotides can be naturally occurring or artificial.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or ribonucleic acid (RNA). The polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA), bridged nucleic acid (BNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety.

The polynucleotide is preferably DNA, RNA or a DNA or RNA hybrid, most preferably DNA. A DNA/RNA hybrid may comprise DNA and RNA on the same strand. Preferably, the DNA/RNA hybrid comprises one DNA strand hybridized to a RNA strand.

The backbone of the polynucleotide can be altered to reduce the possibility of strand scission. For example, DNA is known to be more stable than RNA under many conditions. The backbone of the polynucleotide strand can be modified to avoid damage caused by e.g. harsh chemicals such as free radicals.

DNA or RNA that contains unnatural or modified bases can be produced by amplifying natural DNA or RNA polynucleotides in the presence of modified NTPs using an appropriate polymerase.

The nucleotides in the polynucleotide may be modified. The nucleotides may be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified with a label or a tag.

The polynucleotide may comprise one or more spacers as described further herein.

In the methods provided herein, a double-stranded polynucleotide is dehybridised as it moves in a first direction with respect to a nanopore. The dehybridisation of the double-stranded polynucleotide forms single-stranded polynucleotides.

A single-stranded polynucleotide may contain regions with strong secondary structures, such as hairpins, quadruplexes, or triplex DNA. Structures of these types can be used to further control the movement of the polynucleotide with respect to the nanopore. For example, secondary structures can be used to pause the movement of the polynucleotide through a nanopore, where the polynucleotide briefly pauses in the nanopore upon encountering each successive secondary structure along the strand before they are unwound and translocated. The polynucleotide may reform secondary structures after it has translocated through the nanopore. Such secondary structures can be used to prevent the polynucleotide from moving back through the nanoreactor (nanopore) under low or no applied negative voltages (applied to the trans side of the nanopore) and therefore assist in controlling the movement of the polynucleotide so it only occurs in a controlled manner in the relevant steps of the methods provided herein.

In the provided methods, the polynucleotide being moved is double-stranded. As used herein, a double stranded polynucleotide may comprise single stranded regions and regions with other structures, such as hairpin loops, triplexes and/or quadruplexes. Such secondary structures can be useful as described above in the context of single-stranded polynucleotides.

The two strands of the double-stranded molecule may be covalently linked, for example at the ends of the molecules by joining the 5′ end of one strand to the 3′ end of the other with a hairpin structure.

The polynucleotide can be any length. For example, the polynucleotides can be at least 10, at least 50, at least 100, at least 150, at least 200, at least 250, at least 300, at least 400 or at least 500 nucleotides or nucleotide pairs in length. The target polynucleotide can be 1000 or more nucleotides or nucleotide pairs, 5000 or more nucleotides or nucleotide pairs in length or 100000 or more nucleotides or nucleotide pairs in length or 500,000 or more nucleotides or nucleotide pairs in length, or 1,000,000 or more nucleotides or nucleotide pairs in length, 10,000,000 or more nucleotides or nucleotide pairs in length, or 100,000,000 or more nucleotides or nucleotide pairs in length, or 200,000,000 or more nucleotides or nucleotide pairs in length, or the entire length of a chromosome.

The polynucleotide may be an oligonucleotide. Oligonucleotides are short nucleotide polymers which typically have 50 or fewer nucleotides, such 40 or fewer, 30 or fewer, 20 or fewer, 10 or fewer or 5 or fewer nucleotides. The target oligonucleotide is preferably from about 15 to about 30 nucleotides in length, such as from about 20 to about 25 nucleotides in length. For example, the oligonucleotide can be about 15, about 16, about 17, about 18, about 19, about 20, about 21, about 22, about 23, about 24, about 25, about 26, about 27, about 28, about 29 or about 30 nucleotides in length.

The polynucleotide may be a fragment of a longer target polynucleotide. In this embodiment, the longer polynucleotide is typically fragmented into multiple, such as two or more, shorter polynucleotides.

The target polynucleotide may comprise the products of a PCR reaction, genomic DNA, the products of an endonuclease digestion and/or a DNA library.

The polynucleotide may be naturally occurring. The target polynucleotide may be secreted from cells. Alternatively, the target analyte can be an analyte that is present inside cells such that the analyte must be extracted from the cells before the method can be carried out.

The polynucleotide may be sourced from common organisms such as viruses, bacteria, archaea, plants or animals. Such organisms may be selected or altered to adjust the sequence of the source polynucleotide, for example by adjusting the base composition, removing unwanted sequence elements, and the like. The selection and alteration of organisms in order to arrive at desired polynucleotide characteristics is routine for one of ordinary skill in the art.

The source organism for the polynucleotide may be chosen based on desired characteristics of the sequence. Desired characteristics include the ratio of single-stranded vs double-stranded polynucleotides produced by the organism; the complexity of the sequences of polynucleotides produced by the organism, the composition of the polynucleotides produced by the organism (such as the GC composition), or the length of contiguous polynucleotide strands produced by the organism. For example, when a contiguous polynucleotide strand of around 50 kb is required, lambda phage DNA can be used. If longer contiguous strands are required, other organisms can be used to produce the polynucleotide; for example E. coli produces around 4.5 Mb of contiguous dsDNA.

The polynucleotide is often obtained from a human or animal, e.g. from urine, lymph, saliva, mucus, seminal fluid or amniotic fluid, or from whole blood, plasma or serum. The target polynucleotide may be obtained from a plant e.g. a cereal, legume, fruit or vegetable. The target polynucleotide may comprise genomic DNA. The genomic DNA may be fragmented. The DNA may be fragmented by any suitable method. For example, methods of fragmenting DNA are known in the art, Such methods may use a transposase, such as a MuA transposase. Often the genomic DNA is not fragmented.

In some embodiments—for example when the methods provided herein are used to control the modification of the polynucleotide—the identity of the natural polynucleotide in each nanopore is known by controlling the source material. For example, the source template material could be full length contiguous ˜50 kbase lambda phage dsDNA for each nanoreactor (e.g. nanopore) in a system containing multiple nanoreactors (e.g. nanopores). However, although the starting polynucleotides may be identical in each case, the modifications made to each individual polynucleotide can be different, for example to encode different information into each polynucleotide. In some embodiments the sequence of the polynucleotide being fed through the nanopore is not known in advance. In other embodiments the sequence of the polynucleotide is known.

In some embodiments when using natural polynucleotide sources the strands used are not known before capture in nanopore for modification. In these cases it is sometimes preferable to sequence the polynucleotide either before or during the modification process.

In one embodiment a polynucleotide may be first sequenced before being modified, either in part or in its entirety, then aligned against known reference databases to determine the identity of the sequence. Once the sequence is known the modification process can be altered based on knowledge of the composition. For example, if the modification process only acts on G bases, then knowledge of the sequence can be used to control the position of the strand, for example to control the pausing of the desired sequence containing the G bases in the reaction volume to ensure optimal modification of desired bases.

In another embodiment the strand may be sequenced and/or identified during the process of modification. This can be used to better control which regions of the strand are modified. The methods of the invention can thus be used to control the movement of the polynucleotide strand first to allow its characterisation and then to allow its modification.

In some embodiments a polynucleotide sequence may be determined in real-time by aligning real-time signal or basecalling to known references. Exemplary methods of determining a polynucleotide sequence are described in WO 2016/059427, incorporated by reference herein.

In embodiments which comprise modification of the polynucleotide, pre-determined knowledge of the base composition of the polynucleotide strand can be used to more selectively control when the reaction condition is applied. This can be useful to control the modification to selectively modify single bases or a select number of bases in a known location. In some embodiments the base composition is not known, and the modifications are made in longer stretches that cover multiple bases, creating “islands” of modification and unmodified. Additionally, sequencing can be used to confirm that the modification has been successful. Further actions can then be taken on the strand when unsuccessful modification is detected. For example, the region to be modified can be returned to the reaction volume after unsuccessful modification to repeat the modification process. This process can be repeated until successful modification is detected.

In some embodiments the polynucleotide is synthetic or semi-synthetic. For example, the DNA or RNA may be purely synthetic, synthesised by conventional DNA synthesis methods such as phosphoamidite based chemistries. Synthetic polynucleotides subunits may be joined together by known means, such as ligation or chemical linkage, to produce longer strands. In some embodiments internal self-forming structures (eg. hairpins, quadruplexes) can be designed into the substrate e.g. by ligating appropriate sequences

In some embodiments wherein the methods provided herein are used to modify the polynucleotide strand, the polynucleotide may have a simplified nucleotide composition over a majority of the region where modifications (if made) will be implemented. For example, a DNA strand may be composed of only, or substantially, G and A bases. Such bases do not readily hybridise and can be readily distinguished in modified and unmodified forms. In some embodiments the polynucleotide has a repeating pattern of the same subunit. For example, a repeating unit may be (AmGn)q, wherein m, n and q are positive integers. For example, m is often from 1 to 20, such as from 1 to 10 e.g. from 1 to 5, e.g. 1, 2, 3, 4 or 5. n is often from 1 to 20, such as from 1 to 10 e.g. from 1 to 5, e.g. 1, 2, 3, 4 or 5. m and n may be the same or different. q is often from 1 to about 100,000. A typical repeating unit may be for example (AAAAAAGGGGGG)q.

Repeating polynucleotides can be made by many means known in the art, for example by concatenating together synthetic subunits with sticky ends that enable ligation. In some embodiments the polynucleotide may therefore be a concatenated polynucleotide. Methods of concatenating polynucleotides are described in PCT/GB2017/051493.

Synthetic polynucleotides can be copied and scaled up for production by means known in the art, including PCR, incorporation into bacterial factories, and the like.

In some embodiments the polynucleotide may be altered to either increase or decrease reactivity to external agents. For example, in a mixed base system it can be desirable to control which bases are capable of being modified and which background bases should not be modified. Background bases can be selected so that they have reduced reactivity relative to canonical bases to limit the possibility of unwanted reactions. For example, bases ATC are less reactive than G, so it is possible to adjust the composition of the polynucleotide strand to have runs of less reactive ATC bases in regions where modification is not required, and regions with high G content where modification is to be made. Alternatively, bases that are intended to be modified can be selected to have higher reactivity when exposed to the appropriate reactions conditions.

In some embodiments, the polynucleotide can comprise bases which contain a reactive side-chain. Any suitable reactive functional groups can be incorporated on the side chain as required. Suitable examples of reactive functional groups include click chemistry reagents.

Click chemistry is a term first introduced by Kolb et al. in 2001 to describe an expanding set of powerful, selective, and modular building blocks that work reliably in both small- and large-scale applications (Kolb HC, Finn, MG, Sharpless KB, Click chemistry: diverse chemical function from a few good reactions, Angew. Chem. Int. Ed. 40 (2001) 2004-2021). They have defined the set of stringent criteria for click chemistry as follows: “The reaction must be modular, wide in scope, give very high yields, generate only inoffensive by-products that can be removed by non-chromatographic methods, and be stereospecific (but not necessarily enantioselective). The required process characteristics include simple reaction conditions (ideally, the process should be insensitive to oxygen and water), readily available starting materials and reagents, the use of no solvent or a solvent that is benign (such as water) or easily removed, and simple product isolation. Purification if required must be by non-chromatographic methods, such as crystallization or distillation, and the product must be stable under physiological conditions”.

Suitable examples of click chemistry include, but are not limited to, the following:
(a) copper-free variant of the 1,3 dipolar cycloaddition reaction, where an azide reacts with an alkyne under strain, for example in a cyclooctane ring;
(b) the reaction of an oxygen nucleophile on one linker with an epoxide or aziridine reactive moiety on the other; and
(c) the Staudinger ligation, where the alkyne moiety can be replaced by an aryl phosphine, resulting in a specific reaction with the azide to give an amide bond.

Any reactive group may be used in the invention. The reactive group may be one that is suitable for click chemistry. The reactive group may be any of those disclosed in WO 2010/086602, particularly in Table 4 of that application.

A polynucleotide as used herein is typically stable under appropriate storage conditions for extended periods of time. For example, the polynucleotide may be stable for in excess of 1 day, 1 month, 1 year, 10 years, etc, when stored under appropriate conditions. The necessary stability of a polynucleotide can be determined based on its application and can be controlled using methods known in the art, including the use of high purity reagents and storage under appropriate conditions.

### Polynucleotide Adapter

In some embodiments, the motor protein and/or polynucleotide-handling enzyme if present may be provided on a polynucleotide adapter. WO 2015/110813 describes the loading of motor proteins onto a target polynucleotide such as an adapter, and is hereby incorporated by reference in its entirety.

An adapter typically comprises a polynucleotide strand capable of being attached to the end of a target polynucleotide. The target polynucleotide is typically intended for characterisation or modification in accordance with methods disclosed herein.

A polynucleotide adapter may be added to both ends of the target polynucleotide. Alternatively, different adapters may be added to the two ends of the target polynucleotide. An adapter may be added to just one end of the target polynucleotide. Methods of adding adapters to polynucleotides are known in the art. Adapters may be attached to polynucleotides, for example, by ligation, by click chemistry, by tagmentation, by topoisomerisation or by any other suitable method.

An adapter may be synthetic or artificial. Typically, an adapter comprises a polymer as described herein. In some embodiments, the adapter comprises a polynucleotide. In some embodiments an adapter may comprise a single-stranded polynucleotide strand. In some embodiments an adapter may comprise a double-stranded polynucleotide. A polynucleotide adapter may comprise DNA, RNA, modified DNA (such as a basic DNA), RNA, PNA, LNA, BNA and/or PEG. Usually, the adapter comprises single stranded and/or double stranded DNA or RNA.

An adapter may comprise a spacer as described herein. The adapter may comprise a loading site for a motor protein or polynucleotide-handling enzyme. The adapter may comprise a tag.

An adapter may be a Y adapter. A Y adapter is typically double stranded and comprises (a) at one end, a region where the two strands are hybridised together and (b), at the other end, a region where the two strands are not complementary. The non-complementary parts of the strands form overhangs. The presence of a non-complementary region in the Y adapter gives the adapter its Y shape since the two strands typically do not hybridise to each other unlike the double stranded portion. A motor protein or polynucleotide-handling enzyme may bind to an overhang of an adapter such as a Y adapter. In another embodiment, a motor protein or polynucleotide-handling enzyme may bind to the double stranded region. In other embodiments, a motor protein or polynucleotide-handling enzyme may bind to a single-stranded and/or a double-stranded region of the adapter. In other embodiments, a first motor protein or polynucleotide-handling enzyme may bind to the single-stranded region of such an adapter and a second motor protein or polynucleotide-handling enzyme may bind to the double-stranded region of the adapter.

In one embodiment the adapter comprises a membrane anchor or a pore anchor. In some embodiments, the anchor may be attached to a polynucleotide that is complementary to and hence that is hybridised to the overhang to which a motor protein or polynucleotide-handling enzyme is bound.

In some embodiments, one of the non-complementary strands of a polynucleotide adapter such as a Y adapter may comprise a leader sequence, which when contacted with a transmembrane pore is capable of threading into a nanopore.

The leader sequence typically comprises a polymer such as a polynucleotide, for instance DNA or RNA, a modified polynucleotide (such as abasic DNA), PNA, LNA, polyethylene glycol (PEG) or a polypeptide. In some embodiments, the leader sequence comprises a single strand of DNA, such as a poly dT section. The leader sequence can be any length, but is typically 10 to 150 nucleotides in length, such as from 20 to 120, 30 to 100, 40 to 80 or 50 to 70 nucleotides in length.

In one embodiment, a polynucleotide adapter is a hairpin loop adapter. A hairpin loop adapter is an adapter comprising a single polynucleotide strand, wherein the ends of the polynucleotide strand are capable of hybridising to each other, or are hybridized to each other, and wherein the middle section of the polynucleotide forms a loop. Suitable hairpin loop adapters can be designed using methods known in the art.

As explained in more detail below, a polynucleotide adapter can be attached to a target polynucleotide in order to characterise the target polynucleotide.

Those skilled in the art will also appreciate that when the adapter comprises a polynucleotide strand, the sequence of the adapter is typically not determinative and can be controlled or chosen according to the motor protein and other experimental conditions such as any polynucleotide to be characterised. Exemplary sequences are provided solely by way of illustration in the examples. For example, the adapter may comprise a sequence such as one or more of SEQ ID NOs: 10-12 or a polynucleotide sequence having at least 20%, such as at least 30%, e.g. at least 40% such as at least 50%, e.g. at least 60% such as at least 70%, e.g. at least 80%, for example at least 90% e.g. at least 95% sequence similarity or identity to one or more of SEQ ID NOs: 10-12. The sequence of the adapter can typically be altered without negatively affecting the efficacy of the methods provided herein.

In some embodiments a polynucleotide adapter may comprise a loading site for loading the motor protein and/or polynucleotide-handling enzyme. The loading site may be for instance a single-stranded region which can targeted by the motor protein or polynucleotide-handling enzyme. The loading site may be a region of the polynucleotide adapter to which a exogenous polynucleotide strand comprising the motor protein or polynucleotide-handling enzyme can bind in order to transfer the motor protein or polynucleotide-handling enzyme to the polynucleotide to be assessed in the methods provided herein.

### Spacer(s)

In some embodiments, a polynucleotide or adapter may comprise a spacer. For example, one or more spacers may be present in a polynucleotide adapter. For example, a polynucleotide adapter may comprise from one to about 10 spacers, e.g. from 1 to about 5 spacers, e.g. 1, 2, 3, 4 or 5 spacers. A spacer may comprise any suitable number of spacer units. A spacer provides an energy barrier which impedes movement of a polynucleotide binding protein. For example, a spacer may stall a motor protein or polynucleotide-handling enzyme by reducing the traction of the motor protein or polynucleotide-handling enzyme on the polynucleotide. This may be achieved for instance by using an abasic spacer i.e. a spacer in which the bases are removed from one or more nucleotides in the polynucleotide adapter. A spacer may physically block movement of a motor protein or polynucleotide-handling enzyme, for instance by introducing a bulky chemical group to physically impede the movement of the protein.

In some embodiments, one or more spacers included in the polynucleotide or in an adapter provide a distinctive signal when they pass through or across a nanopore.

In some embodiments, a spacer may comprise a linear molecule, such as a polymer. Typically, such a spacer has a different structure from the target polynucleotide. For instance, if the target polynucleotide is DNA, the or each spacer typically does not comprise DNA. In particular, if the target polynucleotide is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA), bridged nucleic acid (BNA) or a synthetic polymer with nucleotide side chains. In some embodiments, a spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂-C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units. For instance, a spacer may comprise from 1 to about 12 or more (e.g. from about 1 to about 8, for instance from 1 to about 6 such as from 1 to about 4) of such spacer groups.

In some embodiments, a spacer may comprise one or more chemical groups which cause the a motor protein or polynucleotide-handling enzyme to stall. In some embodiments, suitable chemical groups are one or more pendant chemical groups. The one or more chemical groups may be attached to one or more nucleobases in the polynucleotide adapter. The one or more chemical groups may be attached to the backbone of the polynucleotide adapter. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

In some embodiments, a spacer may comprise a polymer. In some embodiments the spacer may comprise a polymer which is a polypeptide or a polyethylene glycol (PEG).

In some embodiments, a spacer may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

Suitable spacers can be designed or selected depending on the nature of the polynucleotide/polynucleotide adapter, the motor protein or polynucleotide-handling enzyme and the conditions under which the method is to be carried out. For example, many polynucleotide processing proteins process DNA in vivo and such polynucleotide binding proteins may typically be stalled using anything that is not DNA.

### Tags

In some embodiments a polynucleotide or polynucleotide adapter may comprise a tag or tether. For example, a polynucleotide can bind to a tag on a nanopore, e.g., via its adaptor, and release at some point, e.g., during characterization of the polynucleotide by the nanopore. A strong non-covalent bond (e.g., biotin/avidin) is still reversible and can be useful in some embodiments of the methods described herein.

In some embodiments, the pair of pore tag and polynucleotide adaptor can be configured such that the binding strength or affinity of a binding site on the polynucleotide (e.g., a binding site provided by an anchor or a leader sequence of an adaptor or by a capture sequence within the duplex stem of an adaptor) to a tag on a nanopore is sufficient to maintain the coupling between the nanopore and polynucleotide until an applied force is placed on it to release the bound polynucleotide from the nanopore.

In some embodiments, the tags or tethers are uncharged. This can ensure that the tags or tethers are not drawn into the nanopore under the influence of a potential difference.

One or more molecules that attract or bind the polynucleotide or adaptor may be linked to the detector (e.g. the pore). Any molecule that hybridizes to the adaptor and/or target polynucleotide may be used. The molecule attached to the pore may be selected from a PNA tag, a PEG linker, a short oligonucleotide, a positively charged amino acid and an aptamer. Pores having such molecules linked to them are known in the art. For example, pores having short oligonucleotides attached thereto are disclosed in Howarka et al (2001) Nature Biotech. 19: 636-639 and WO 2010/086620, and pores comprising PEG attached within the lumen of the pore are disclosed in Howarka et al (2000) J. Am. Chem. Soc. 122(11): 2411-2416.

A short oligonucleotide attached to the detector (e.g. a transmembrane pore), which oligonucleotide comprises a sequence complementary to a sequence in the leader sequence or another single stranded sequence in the adaptor may be used to enhance capture of the target polynucleotide in the methods described herein.

In some embodiments, the tag or tether may comprise or be an oligonucleotide (e.g., DNA, RNA, LNA, BNA, PNA, or morpholino). The oligonucleotide (e.g., DNA, RNA, LNA, BNA, PNA, or morpholino) can have about 10-30 nucleotides in length or about 10-20 nucleotides in length. In some embodiments, the oligonucleotide (e.g., DNA, RNA, LNA, BNA, PNA, or morpholino) for use in the tag or tether can have at least one end (e.g., 3′- or 5′-end) modified for conjugation to other modifications or to a solid substrate surface including, e.g., a bead. The end modifiers may add a reactive functional group which can be used for conjugation. Examples of functional groups that can be added include, but are not limited to amino, carboxyl, thiol, maleimide, aminooxy, and any combinations thereof. The functional groups can be combined with different length of spacers (e.g., C3, C9, C12, Spacer 9 and 18) to add physical distance of the functional group from the end of the oligonucleotide sequence.

In some embodiments, the tag or tether may comprise or be a morpholino oligonucleotide. The morpholino oligonucleotide can have about 10-30 nucleotides in length or about 10-20 nucleotides in length. The morpholino oligonucleotides can be modified or unmodified. For example, in some embodiments, the morpholino oligonucleotide can be modified on the 3′ and/or 5′ ends of the oligonucleotides. Examples of modifications on the 3′ and/or 5′ end of the morpholino oligonucleotides include, but are not limited to 3′ affinity tag and functional groups for chemical linkage (including, e.g., 3′-biotin, 3′-primary amine, 3′-disulfide amide, 3′-pyridyl dithio, and any combinations thereof); 5′ end modifications (including, e.g., 5′-primary ammine, and/or 5′-dabcyl), modifications for click chemistry (including, e.g., 3′-azide, 3′-alkyne, 5′-azide, 5′-alkyne), and any combinations thereof.

In some embodiments, the tag or tether may further comprise a polymeric linker, e.g., to facilitate coupling to a detector e.g. a nanopore. An exemplary polymeric linker includes, but is not limited to polyethylene glycol (PEG). The polymeric linker may have a molecular weight of about 500 Da to about 10 kDa (inclusive), or about 1 kDa to about 5 kDa (inclusive). The polymeric linker (e.g., PEG) can be functionalized with different functional groups including, e.g., but not limited to maleimide, NHS ester, dibenzocyclooctyne (DBCO), azide, biotin, amine, alkyne, aldehyde, and any combinations thereof. In some embodiments, the tag or tether may further comprise a 1 kDa PEG with a 5′-maleimide group and a 3′-DBCO group. In some embodiments, the tag or tether may further comprise a 2 kDa PEG with a 5′-maleimide group and a 3′-DBCO group. In some embodiments, the tag or tether may further comprise a 3 kDa PEG with a 5′-maleimide group and a 3′-DBCO group. In some embodiments, the tag or tether may further comprise a 5 kDa PEG with a 5′-maleimide group and a 3′-DBCO group.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of a nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444.

In some embodiments where one or more tags or tethers are attached to a nanopore via cysteine linkage(s), the one or more cysteines can be introduced to one or more monomers that form the nanopore by substitution. In some embodiments, the nanopore may be chemically modified by attachment of (i) Maleimides including diabromomaleimides such as: 4-phenylazomaleinanil, 1.N-(2-Hydroxyethyl)maleimide, N-Cyclohexylmaleimide, 1.3-Maleimidopropionic Acid, 1.1-4-Aminophenyl-1H-pyrrole,2,5,dione, 1.1-4-Hydroxyphenyl-1H-pyrrole,2,5,dione, N-Ethylmaleimide, N-Methoxycarbonylmaleimide, N-tert-Butylmaleimide, N-(2-Aminoethyl)maleimide, 3-Maleimido-PROXYL, N-(4-Chlorophenyl)maleimide, 1-[4-(dimethylamino)-3,5-dinitrophenyl]-1H-pyrrole-2,5-dione, N-[4-(2-Benzimidazolyl)phenyl]maleimide, N-[4-(2-benzoxazolyl)phenyl]maleimide, N-(1-naphthyl)-maleimide, N-(2,4-xylyl)maleimide, N-(2,4-difluorophenyl)maleimide, N-(3-chloro-para-tolyl)-maleimide, 1-(2-amino-ethyl)-pyrrole-2,5-dione hydrochloride, 1-cyclopentyl-3-methyl-2,5-dihydro-1H-pyrrole-2,5-dione, 1-(3-aminopropyl)-2,5-dihydro-1H-pyrrole-2,5-dione hydrochloride, 3-methyl [2-oxo-2-(piperazin-1-yl)ethyl]-2,5-dihydro-1H-pyrrole-2,5-dione hydrochloride, 1-benzyl-2,5-dihydro-1H-pyrrole-2,5-dione, 3-methyl-1-(3,3,3-trifluropropyl)-2,5-dihydro-1H-pyrrole-2,5-dione, 1-[4-(methylamino)cyclohexyl]-2,5-dihydro-1H-pyrrole-2,5-dione trifluroacetic acid, SMILES O═C1C═CC(═O)N1CC═2C═CN═CC2, SMILES O═C1C═CC(═O)N1CN2CCNCC2, 1-benzyl-3-methyl-2,5-dihydro-1H-pyrrole-2,5-dione, 1-(2-fluorophenyl)-3-methyl-2,5-dihydro 1H-pyrrole-2,5-dione, N-(4-phenoxyphenyl)maleimide, N-(4-nitrophenyl)maleimide (ii) Iodocetamides such as :3-(2-Iodoacetamido)-proxyl, N-(cyclopropylmethyl)-2-iodoacetamide, 2-iodo-N-(2-phenylethyl)acetamide, 2-iodo-N-(2,2,2-trifluoroethyl)acetamide, N-(4-acetylphenyl)-2-iodoacetamide, N-(4-(aminosulfonyl)phenyl)-2-iodoacetamide, N-(1,3-benzothiazol-2-yl)-2-iodoacetamide, N-(2,6-diethylphenyl)-2-iodoacetamide, N-(2-benzoyl-4-chlorophenyl)-2-iodoacetamide, (iii) Bromoacetamides: such as N-(4-(acetylamino)phenyl)-2-bromoacetamide, N-(2-acetylphenyl)-2-bromoacetamide, 2-bromo-n-(2-cyanophenyl)acetamide, 2-bromo-N-(3-(trifluoromethyl)phenyl)acetamide, N-(2-benzoylphenyl)-2-bromoacetamide, 2-bromo-N-(4-fluorophenyl)-3-methylbutanamide, N-Benzyl-2-bromo-N-phenylpropionamide, N-(2-bromo-butyryl)-4-chloro-benzenesulfonamide, 2-Bromo-N-methyl-N-phenylacetamide, 2-bromo-N-phenethyl-acetamide,2-adamantan-1-yl-2-bromo-N-cyclohexyl-acetamide, 2-bromo-N-(2-methylphenyl)butanamide, Monobromoacetanilide, (iv) Disulphides such as: aldrithiol-2, aldrithiol-4, isopropyl disulfide, 1-(Isobutyldisulfanyl)-2-methylpropane, Dibenzyl disulfide, 4-aminophenyl disulfide, 3-(2-Pyridyldithio)propionic acid, 3-(2-Pyridyldithio)propionic acid hydrazide, 3-(2-Pyridyldithio)propionic acid N-succinimidyl ester, am6amPDP1-(3CD and (v) Thiols such as: 4-Phenylthiazole-2-thiol, Purpald, 5,6,7,8-tetrahydro-quinazoline-2-thiol.

In some embodiments, the tag or tether may be attached directly to a nanopore or via one or more linkers. The tag or tether may be attached to the nanopore using the hybridization linkers described in WO 2010/086602. Alternatively, peptide linkers may be used. Peptide linkers are amino acid sequences. The length, flexibility and hydrophilicity of the peptide linker are typically designed such that it does not to disturb the functions of the monomer and pore. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

### Anchor

In one embodiment, a polynucleotide or polynucleotide adapter may comprise a membrane anchor or a transmembrane pore anchor. In one embodiment the anchor assists in the characterisation of a target polynucleotide in accordance with the methods disclosed herein. For example, a membrane anchor or transmembrane pore anchor may promote localisation of the selected polynucleotides around a nanopore.

The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol. The anchor may comprise thiol, biotin or a surfactant. In one aspect the anchor may be biotin (for binding to streptavidin), amylose (for binding to maltose binding protein or a fusion protein), Ni-NTA (for binding to poly-histidine or poly-histidine tagged proteins) or peptides (such as an antigen).

In one embodiment, the anchor comprises a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The adapter may hybridise to a complementary sequence on a circular polynucleotide linker. The one or more anchors or one or more linkers may comprise a component that can be cut or broken down, such as a restriction site or a photolabile group. The linker may be functionalised with maleimide groups to attach to cysteine residues in proteins. Suitable linkers are described in WO 2010/086602.

In one embodiment, the anchor is cholesterol or a fatty acyl chain. For example, any fatty acyl chain having a length of from 6 to 30 carbon atom, such as hexadecanoic acid, may be used. Examples of suitable anchors and methods of attaching anchors to adapters are disclosed in WO 2012/164270 and WO 2015/150786.

In another embodiment the anchor may consist or comprise a hydrophobic modification to the polynucleotide or polynucleotide adapter. The hydrophobic modification may comprise a modified phosphate group comprised within the polynucleotide or polynucleotide anchor. The hydrophobic modification may for example comprise a phosphorothioate such as a charge-neutralized alkyl-phosphorothioate (PPT) as described in Jones et al, J. Am. Chem. Soc. 2021, 143, 22, 8305, the entire contents of which are hereby incorporated by reference. Suitable alkyl groups include for example C₁-C₁₀ alkyl groups such as C₂-C₆ alkyl groups; e.g. methyl, ethyl, propyl, butyl, pentyl and hexyl groups. Incorporation of the charge-neutralized alkyl-phosphorothioate into a polynucleotide allows for the polynucleotide to anchor to a hydrophobic region such as a lipid bilayer.

### Characterising

In some embodiments, the provided methods relate to characterising a target polynucleotide.

The characterisation methods typically comprise taking one or more measurements as the double-stranded polynucleotide moves with respect to the detector such as a nanopore in accordance with the methods provided herein, wherein the one or more measurements are indicative of one or more characteristics of the polynucleotide, and thereby characterising the polynucleotide as it moves with respect to the nanopore.

Accordingly, provided herein is a method of characterising a target double-stranded polynucleotide analyte, comprising:

a) contacting the polynucleotide with a motor protein and a nanopore;

b1) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;

b2) taking one or more or more measurements indicative of one or more characteristics of the target polynucleotide as the double stranded polynucleotide moves in the first direction with respect to the nanopore;

c1) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises;

c2) optionally taking one or more or more measurements indicative of one or more characteristics of the target polynucleotide as the double stranded polynucleotide moves in the second direction with respect to the nanopore;

d1) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore; and

d2) taking one or more or more measurements indicative of one or more characteristics of the target polynucleotide as the double stranded polynucleotide moves in the first direction with respect to the nanopore;

wherein the active double stranded polynucleotide-unwinding activity of the motor protein is suppressed.

In some embodiments, the method further comprises:

e1) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises; and

e2) optionally taking one or more or more measurements indicative of one or more characteristics of the target polynucleotide as the double stranded polynucleotide moves in the second direction with respect to the nanopore.

Features of such methods such as the nature of the polynucleotide, motor protein, nanopore, forces applied etc are generally as described herein for the methods of moving a polynucleotide.

### Modification

As explained above, in some embodiments the provided methods are used in methods of encoding data on a double-stranded polynucleotide strand. In such methods, one or more of the steps (b), (c), (d) and (e) if present typically comprise modifying the portion of the polynucleotide in the vicinity of the nanopore as the polynucleotide moves with respect to the nanopore.

In one embodiment, therefore, provided is a method of encoding data on a double-stranded polynucleotide, comprising:

a) contacting the polynucleotide with a motor protein and a nanopore;

b1) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;

b2) modifying the portion of the polynucleotide in the vicinity of the nanopore as the double stranded polynucleotide moves in the first direction with respect to the nanopore;

c1) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises;

c2) optionally modifying the portion of the polynucleotide in the vicinity of the nanopore as the double stranded polynucleotide moves in the second direction with respect to the nanopore;

d1) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore; and

d2) modifying the portion of the polynucleotide in the vicinity of the nanopore as the double stranded polynucleotide moves in the first direction with respect to the nanopore;

wherein the active double stranded polynucleotide-unwinding activity of the motor protein is suppressed.

In some embodiments, the method further comprises:

e1) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises; and

e2) optionally modifying the portion of the polynucleotide in the vicinity of the nanopore as the double stranded polynucleotide moves in the second direction with respect to the nanopore.

Features of such methods such as the nature of the polynucleotide, motor protein, nanopore, forces applied etc are generally as described herein for the methods of moving a polynucleotide.

In embodiments of the provided methods which comprise modifying the double-stranded polynucleotide, the number of nucleotides that are modified within each portion of the polynucleotide strand is typically controlled. For example, in some embodiments 1 nucleotide may be selectively modified. In other embodiments more than one nucleotide may be selectively modified; for example from 1 to about 1000 nucleotides, such as from about 10 to about 500, e.g. from about 50 to about 250 nucleotides may be selectively modified. Different portions of the polynucleotide strand may have different numbers of modified nucleotides and thereby be distinguished.

In other embodiments the methods comprise selectively controlling the extent of the modifications made to the nucleotides that are modified within each portion of the polynucleotide strand. For example, in some embodiments extensive modifications may be made to nucleotides in the vicinity of the nanopore when the portion is within such vicinity for a prolonged time period, whereas other portions of the polynucleotide strand may be within the nanopore for a shorter period and be less extensively modified. Different portions of the polynucleotide strand may thus be modified to different extents and thereby by distinguished.

Typically in such methods the polynucleotide is within a nanovolume extending to about 30 nm from one or more openings of the nanopore. For example, the nanovolume may comprise a volume extending to about 20 nm, about 10 nm or about 5 nm from one or more openings of the nanopore. If a nanopore has both a cis and a trans opening, for example, the nanovolume may extend from the cis opening, the trans opening or both the cis and the trans opening.

In some embodiments, selectively modifying portions of the polynucleotide strand comprises (i) scission of part of the polynucleotide strand, e.g. scission of one or more side chains on the polynucleotide strand; (ii) modification of the strand, e.g. modification of one or more side chains on the strand; and/or (iii) addition to the strand, e.g. additional of pendant chemical groups to the backbone or sidechains of the residues on the polynucleotide strand. When selectively modifying the polynucleotide strand comprises a scission reaction, the reaction is typically not scission of the polynucleotide backbone such that the overall strand length remains unaltered by the modification reaction.

In some embodiments, modifying the polynucleotide comprises subjecting the portion of the polynucleotide in the vicinity of the nanopore to reaction conditions comprising (i) the presence, absence or concentration of one or more chemical reagent(s); (ii) the engagement of an enzyme with the polynucleotide strand under conditions that the enzyme modifies the nucleotides within the polynucleotide strand; (iii) the presence or absence of electromagnetic radiation; and/or (iv) the presence or absence of applied heat.

In some embodiments, modification of the polynucleotide strand can be controlled by controlling the presence, absence or concentration of one or more chemical reagent(s) by applying an electrical or chemical potential across the nanopore. For example, when charged reagents are used then a chemical potential can be used to control the movement of such species into the nanopore and thus to control the concentration of such reagents in the vicinity of the nanopore. Ionic flux conditions through a nanopore can be altered by changing the applied voltage, so a chemical potential or voltage control can be used regardless of whether the chemical reagents are charged or not. Both a chemical and electrical potential can be used.

Typically, such chemical reagents comprise at least a first reagent and a second reagent. The first and second reagents react with the portion of the polynucleotide strand in the vicinity of the nanopore. However, the methods is not limited to embodiments in which multiple reagents are used. The methods may involve the use of only one chemical reagent to modify the polynucleotide, or multiple reagents may be used. In an exemplary embodiment two reagents are used.

There are many suitable combinations of reagents which react to modify polynucleotides, such as combinations of metal ions and oxidising agents such as peroxide etc. In the provided methods, any suitable modification can be detected. For example, the modification can be made to the backbone, the sugar, and/or the bases of polynucleotides in the nanoreactor.

In some embodiments the polynucleotide is modified by a click chemical reaction. In some embodiments the click reaction is between a reactive group on the polynucleotide and a reactive molecule in solution or attached to the nanopore. Click chemistry conducted within nanopore nanoreactors has been described in e.g. Haugland et al, “Synthetically Diversified Protein Nanopores: Resolving Click Reaction Mechanisms” ACS Nano 2019. Examples of click chemical reactions and reagents are described in more detail herein.

In the provided methods, it is typically desired that the polynucleotide external to the nanopore is not modified whereas the portion of the polynucleotide within the nanopore is modified as described herein. This can be achieved by localising chemical reagents in the nanopore. Localisation of chemical reagents in the nanopore can be achieved by providing the reagents precisely to the nanopore or by synthesizing the reagents in nanopore. Localisation of chemical reagents in the nanopore can also be achieved by the use of protectants to remove the chemical reagents from the environment external to the nanopore. One or more protectant(s) is thus typically provided external to the nanopore to minimise or prevent modification of the polynucleotide by chemical reagent(s) external to the nanopore.

Suitable protectants are known in the art and any suitable protectant can be used, depending on the nature of the chemical reagent which it is desired to target, for example chelating agents, antioxidants, oxygen scavengers, reducing agents, etc. Particularly suitable protectants include chelating agents. Suitable chelating agents are known for a wide variety of chemical reagents including both cations and anions. For example, chelating agents such as EDTA are useful in removing metal ions from the environments external to a nanopore (e.g. from the environments outside the internal cavity of a nanopore). Other suitable chelating agents include vitamin B12, citric acid, crown ethers, calixarenes, and the like.

When a protectant is used in the disclosed methods, the protectant is typically used in excess relative to the amount of the chemical reagent at issue. The protectant is often present in an amount at least twice, such as at least 5 times, at least 10 times, at least 100 times, or at least 1000 times the concentration of the chemical reagent at issue.

The protectant is often located in a different compartment of a system to the bulk of the chemical reagent it is intended to sequester. For example, in methods in which metal ions are used to modify the polynucleotide, the metal ions are often located trans of the nanopore, such that when a positive voltage is applied to the trans side of the nanopore the metal ions flow from the trans compartment to the cis compartment through the nanopore. In such cases, a protectant such as EDTA is typically present in the cis compartment in order to sequester metal ions that flow through the nanopore such that polynucleotide in the cis compartment is not modified apart from when it is within the nanopore.

In some embodiments a chemical sensitizer is used to facilitate the redox modification of the polynucleotide strand in the nanopore. In some embodiments a redox mediator is used as the chemical sensitizer. Examples include peroxide, ascorbate, etc.

In other embodiments, the polynucleotide may be modified by using a polynucleotide-modifying enzyme. Many different polynucleotide-modifying enzymes are known in the art and are suitable for use in the methods disclosed herein. Many polynucleotide modifying enzymes are polynucleotide-handling enzymes and/or motor proteins as discussed herein.

Typically, in embodiments which use a polynucleotide-modifying enzyme, the polynucleotide-modifying enzyme modifies the base portion of nucleotides within the polynucleotide strand. However, the modification can be made to the “backbone” of the polynucleotide strand e.g. to the phosphodiester linkages and/or to the sugars.

In some embodiments, modification of the polynucleotide by the polynucleotide-modifying enzyme is controlled by controlling the presence, absence or concentration of fuel and/or substrate for the enzyme. For example, for an NTP-driven nucleic acid modifying enzyme controlling the concentration of NTPs (e.g. ATP) in the vicinity of the nanopore can allow the modification of the polynucleotide by the polynucleotide-processing enzyme.

Some polynucleotide-modifying enzymes respond to conformational changes induced with physical force is exerted on the polynucleotide substrate. The modification or otherwise of the polynucleotide can thus be controlled by controlling a force exerted on the polynucleotide. Suitable forces include electrophoretic forces applied by applying a voltage across the nanopore. Varying the voltage applied leads to a variation in the force applied on the polynucleotide-modifying enzyme and controls the activity of the enzyme in modifying the polynucleotide.

Some polynucleotide modifying enzymes function selectively on either only single- or double-stranded polynucleotide. As explained, the methods provided herein control the hybridised structure of the polynucleotide and thus can enable selective binding and modification when desired. For example, a DNA strand translocating through a nanopore can be paused as desired long enough for a short secondary polynucleotide to hybridise to form a dsDNA region, to which a ds-DNA selective enzyme can bind to modify the DNA (eg. a DNA-methyltransferase that modifies specific DNA bases).

In some embodiments the methods comprise contacting the portions of the polynucleotide strand in the vicinity of the nanopore with electromagnetic radiation in the form of light, preferably visible or ultraviolet light. Light may be applied using any suitable means. Fibre optics may be used to deliver the light. A laser may be used to irradiate the nanopore.

In some embodiments the methods involve irradiating a photosensitizer for modification of the polynucleotide strand. Any suitable photosensitizer can be used. Suitable photosensitizers include porphyrins and the like. In some embodiments the sensitizer is a light-excited molecule. In some embodiments the sensitizer is stable under excitation.

In some embodiments the sensitizer is an inorganic based nanoparticle or organic based dye. The sensitizer may be chosen for its specific properties including: size, excitation wavelengths, emission wavelengths, reactive properties upon exposure, surface chemistry, etc. The sensitizer may be any re-emitting light source known in the art, that when illuminated globally will re-emit light locally. Well known examples include organic dyes and fluorophores, particles such nanodots, etc.

In some embodiments the methods involve irradiating a sensitizer and transferring radiation from the sensitizer to the polynucleotide strand for modification of the polynucleotide strand. For example, the sensitizer may be a metal nanoparticle, and/or wherein said radiation is electromagnetic radiation or thermal radiation.

In some embodiments a photosensitizer may comprise a redox enzyme or cofactor thereof. In some embodiments a photosensitizer may comprise a nanoparticle. In some embodiments a photosensitizer may comprise an organic or inorganic dye. Suitable dyes include acridine orange, methylene blue, etc.

In some embodiments the sensitizer is an organic molecule such as riboflavin.

In some embodiments the sensitizer is attached to the nanopore. In some embodiments the sensitizer is attached to the outer portions of the nanopore, thus in the vicinity of polynucleotide entering or exiting the nanopore. In some embodiments the sensitizer is attached to an internal surface of the nanopore, e.g. a surface of the channel running through the nanopore. This can be beneficial as it increasing the chance of selectively modifying only the photocleavable element nearby and not other photocleavable elements positioned along the typically very flexible polynucleotide molecules that might otherwise transiently come near to the sensitizer unless occluded. An additional advantage of placing the sensitizer inside the nanopore is that the reaction can occur at or near a reader if placed correctly, so that a change in current upon modification can be detected in situ.

In some embodiments the sensitizer is placed near the entrance or exit to the nanopore, or in constriction regions. For example, many biologically derived nanopores with a wide range of internal dimensions are known, with internal cavities ranging from <1 nm to >10 nm in diameter. Well known examples of include alpha-hemolysin, MspA, CsgG, Aerolysin, Phi29 portal, etc. Likewise a range of small sensitizers are well known in the art (typically <10 nm, preferably 1-4 nm in diameter).

In some embodiments the sensitizer is attached directly to the nanopore, e.g. by covalent attachment to reactive amino-acids, for example employing disulphide or maleimide chemistry.

In some embodiments the sensitizer is a nanoparticle and is attached to the nanopore. Attachment of nanoparticles to alpha-hemolysin is demonstrated in Reiner et al, J. Am. Chem. Soc., 27 (135) 2013. Internalisation of small nanoparticles in nanopores is demonstrated in Chavis et al, ACS Sensors, 2017 (doi: 10.1021/acssensors.7b00362)

In some embodiments the sensitizer is attached to the nanopore in a position to engage with a photocleavable group on the polynucleotide strand being modified. For example, in some embodiments the sensitizer is positioned within about 10 nm, e.g. within about 5 nm, such as within about 3 nm, e.g. within about 2 nm, such as within about 1 nm of a photocleavable group on the strand.

In some embodiments the sensitizer is attached to the nanopore, by maleimide chemistry, e.g. by attachment to cysteines in a nanopore. For example, in some embodiments the nanoreactor is a CsgG nanopore and the sensitizer is conjugated to a cysteine mutation introduced at a suitable residue in the CsgG nanopore. Suitable positions on other protein nanopores can also be readily identified by those skilled in the art.

The modification of the polynucleotide in the vicinity of the nanopore can be achieved using heat. The heat can be applied using any suitable means. For example, local heat can be applied by transferring heat to the polynucleotide from an irradiated sensitizer. The sensitizer may be a plasmonic guide, such as a gold nanoparticle linked to the nanopore. Plasmonic guides can be used enhance temperature changes in a localised region when a system is illuminated over a broad area. When illuminated a plasmonic guide creates a localised heating (within a few 10s of nanometers of the plasmonic guide), while the ambient temperature of the rest of the bulk environment is not significantly altered. When the light is switched off the local temperature increase quickly returns to ambient (often in less a few seconds) as the heat diffuses away. Plasmonic guides linked to or near to a nanopores or motor can therefore be used to alter the local temperature, e.g. by up to tens of degrees. The control of the local temperature in the nanoreactor can thus be used to control the modification of polynucleotides within the nanoreactor.

In some embodiments an array of nanopores may be globally subjected to reaction conditions to modify the portions of the polynucleotide within each nanopore in the array. The global reaction conditions might include for example (as described in more detail herein) irradiation with light, heating, contact with one or more chemical reagents (e.g. by flushing a flow cell with buffer containing reactive components). The duration and pattern of each reaction exposure can be varied to control the pattern of modifications.

In some embodiments the reaction condition is a pulse of irradiation at a specific frequency. For example, as described in more detail herein, a light pulse may be used to excite a sensitizer on the nanopore that re-emits radiation locally to cleave an adjacent photocleavable side-chain on the DNA unit in the nanopore.

### Detector

In the methods provided herein, the polynucleotide is moved with respect to a detector such as a nanopore. The detector may be selected from (i) a zero-mode waveguide, (ii) a field-effect transistor, optionally a nanowire field-effect transistor; (iii) an AFM tip; (iv) a nanotube, optionally a carbon nanotube; and (v) a nanopore. Preferably, the detector is a nanopore.

The polynucleotide may be characterised in the methods provided herein in any suitable manner. In one embodiment the polynucleotide is characterised by detecting an ionic current or optical signal as the polynucleotide moves with respect to a nanopore. This is described in more detail herein. The method is amenable to these and other methods of detecting polynucleotides.

In another non-limiting example, in one embodiment the polynucleotide is characterised by detecting the by-products of a polynucleotide-processing reaction, such as a sequencing by synthesis reaction. The method may thus involve detecting the product of the sequential addition of (poly)nucleotides by an enzyme such as a polymerase to the nucleic acid strand. The product may be a change in one or more properties of the enzyme such as in the conformation of the enzyme. Such methods may thus comprise subjecting an enzyme such as polymerase or a reverse transcriptase to a double-stranded polynucleotide under conditions such that the template-dependent incorporation of nucleotide bases into a growing oligonucleotide strand causes conformational changes in the enzyme in response to sequentially encountering template strand nucleic acid bases and/or incorporating template-specified natural or analog bases (i.e., an incorporation event), detecting the conformational changes in the enzyme in response to such incorporation events, and thereby detecting the sequence of the template strand. In such methods the polynucleotide strand may be moved in accordance with the methods provided herein. Such methods may involve detecting and/or measuring incorporation events using methods known to those skilled in the art, such as those described in US 2017/0044605.

In another embodiment, by-products may be labelled so that a phosphate labelled species is released upon the addition of a nucleotide to a synthesised nucleic acid strand that is complementary to the template strand, and the phosphate labelled species is detected e.g. using a detector as described herein. The polynucleotide being characterised in this way may be moved in accordance with the methods herein. Suitable labels may be optical labels that are detected using a nanopore, or a zero mode wave guide, or by Raman spectroscopy, or other detectors. Suitable labels may be non-optical labels that are detected using a nanopore, or other detectors.

In another approach, nucleoside phosphates (nucleotides) are not labelled and upon the addition of a nucleotide to a synthesised nucleic acid strand that is complementary to the template strand, a natural by-product species is detected. Suitable detectors may be ion-sensitive field-effect transistors, or other detectors.

These and other detection methods are suitable for use in the methods described herein. Any suitable measurements can be taken using a detector as the polynucleotide moves with respect to the detector.

### Nanopore

In embodiments of the disclosed methods wherein the detector is a nanopore. any suitable nanopore can be used. In one embodiment a nanopore is a transmembrane pore.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

Any transmembrane pore may be used in the methods provided herein. The pore may be biological or artificial. Suitable pores include, but are not limited to, protein pores, polynucleotide pores and solid state pores. The pore may be a DNA origami pore (Langecker et al., Science, 2012; 338: 932-936). Suitable DNA origami pores are disclosed in WO2013/083983.

In one embodiment, the nanopore is a transmembrane protein pore. A transmembrane protein pore is a polypeptide or a collection of polypeptides that permits hydrated ions, such as polynucleotide, to flow from one side of a membrane to the other side of the membrane. In the methods provided herein, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

In one embodiment, the nanopore is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

In one embodiment, the transmembrane protein pore comprises a barrel or channel through which the ions may flow. The subunits of the pore typically surround a central axis and contribute strands to a transmembrane β-barrel or channel or a transmembrane α-helix bundle or channel.

Typically, the barrel or channel of the transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

In one embodiment, the nanopore is a transmembrane protein pore derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and a outer membrane proteins, such as WZA and ClyA toxin.

In one embodiment the nanopore is a transmembrane pore derived from or based on Msp, α-hemolysin (α-HL), lysenin, CsgG, ClyA, Spl or haemolytic protein fragaceatoxin C (FraC).

In one embodiment, the nanopore is a transmembrane protein pore derived from CsgG, e.g. from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591.

In one embodiment, the nanopore is a transmembrane pore derived from lysenin. Examples of suitable pores derived from lysenin are disclosed in WO 2013/153359.

In one embodiment, the nanopore is a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

In one embodiment, the nanopore is a transmembrane protein pore derived from Msp, e.g. from MspA. Examples of suitable pores derived from MspA are disclosed in WO 2012/107778.

In one embodiment, the nanopore is a transmembrane pore derived from or based on ClyA.

### Membrane

In the disclosed methods, the nanopore is typically present in a membrane. Any suitable membrane may be used.

The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

In some embodiments, the membrane is one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In some embodiments, a lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The methods disclosed herein are typically carried out using (i) an artificial amphiphilic layer comprising a pore, (ii) an isolated, naturally-occurring lipid bilayer comprising a pore, or (iii) a cell having a pore inserted therein. The methods are typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### General Methods

As mentioned above, the methods provided herein may be operated using any suitable detector, and as such any suitable apparatus for detecting polynucleotides can be used.

The methods provided herein may in some embodiments be carried out using any apparatus that is suitable for transmembrane pore sensing. For example, the apparatus may comprise a chamber comprising an aqueous solution and a barrier that separates the chamber into two sections. The barrier may have an aperture in which a membrane containing a transmembrane pore is formed. Transmembrane pores are described herein.

The methods may be carried out using the apparatus described in WO 2008/102120, WO 2010/122293 or WO 00/28312. In brief. the binding of a molecule (e.g. a target polynucleotide) in the channel of a pore will have an effect on the open-channel ion flow through the pore, which is the essence of “molecular sensing” of pore channels. Variation in the open-channel ion flow can be measured using suitable measurement techniques by the change in electrical current. The degree of reduction in ion flow, as measured by the reduction in electrical current, is related to the size of the obstruction within, or in the vicinity of, the pore. Binding of a molecule of interest (e.g. the target polynucleotide) in or near the pore therefore provides a detectable and measurable event, thereby forming the basis of a “biological sensor”. Detecting the presence of biological molecules finds application in personalised drug development, medicine, diagnostics, life science research, environmental monitoring and in the security and/or the defence industry.

When used to characterize the polynucleotide, the presence, absence or one or more characteristics of the target polynucleotide are determined. The methods may be for determining the presence, absence or one or more characteristics of at least one target polynucleotide. The methods may concern determining the presence, absence or one or more characteristics of two or more target polynucleotide. The methods may comprise determining the presence, absence or one or more characteristics of any number of target polynucleotides, such as 2, 5, 10, 15, 20, 30, 40, 50, 100 or more target polynucleotides. Any number of characteristics of the one or more target polynucleotides may be determined, such as 1, 2, 3, 4, 5, 10 or more characteristics. Characteristics amenable to being detected in the methods provide herein include the identity or sequence of the polynucleotide, the length, of the polynucleotide, whether or not the polynucleotide is modified, etc.

When used to characterize the polynucleotide, the methods may involve measuring the ion current flow through the pore, typically by measurement of a current. Alternatively, the ion flow through the pore may be measured optically, such as disclosed by Heron et al: J. Am. Chem. Soc. 9 Vol. 131, No. 5, 2009. Therefore the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The methods may involve measuring an optical signal as described in Chen et al, Nature Communications (2018) 9:1733, the entire contents of which are hereby incorporated by reference. For example, a nanopore such as an optically engineered nanopore structure (e.g. a plasmonic nanoslit) may be used to locally enable single-molecule surface enhanced Raman spectroscopy (SERS) to allow the characterisation of the polynucleotide through direct Raman spectroscopic detection.

The methods may be carried out on a silicon-based array of wells where each array comprises 128, 256, 512, 1024, 2000, 3000, 4000, 6000, 10000, 12000, 15000 or more wells.

The methods may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

As explained above, in some embodiments the methods provided herein comprise applying a first force and/or a second force across the nanopore, and the first force and/or the second force may comprise a voltage potential. Typically, the first force may be greater than the second force.

In some embodiments the first force is a voltage potential of from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV.

In some embodiments the second force is a voltage potential from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV.

In some embodiments the first force is a voltage potential of from +100 mV to +300 mV, more preferably in the range of +120 mV to +220 mV such as from +150 mV to +200 mV, e.g. +180 mV; and the second force is a voltage potential of from −50 to +100 mV such as from −20 mV to +80 mV, e.g. from 0 mV to about +50 mV such as +20 mV.

The methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt may be an alkaline earth metal salt such as calcium chloride (CaCl2). The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of binding/no binding to be identified against the background of normal current fluctuations.

The methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

### Polynucleotide Adapters

Also provided are polynucleotide adapters comprising motor proteins and polynucleotide-handling enzymes. It will be understood that any of the polynucleotide adapters disclosed herein can be applied in the embodiments of the methods discussed herein and above.

In one embodiment, provided herein is a polynucleotide adapter having a motor protein and a polynucleotide-handling enzyme bound thereto, wherein the motor protein is capable of controlling the movement of the target polynucleotide with respect to a nanopore in a first direction; the polynucleotide-handling enzyme is capable of applying a force to move the target polynucleotide with respect to the nanopore in a second direction opposite to the first direction; and wherein the active double stranded polynucleotide-unwinding activity of the motor protein is suppressed.

In one embodiment, the polynucleotide adapter is a polynucleotide adapter as described in more detailed herein. In one embodiment, the motor protein is a motor protein as described herein. In one embodiment the polynucleotide-handling enzyme is a blocking moiety as described herein.

In one embodiment the motor protein is oriented on the polynucleotide adapter to control the movement of the target polynucleotide with respect to a detector such as a nanopore in a first direction. The first direction may for instance be in the cis-to-trans direction. The motor protein may thus be oriented to process the polynucleotide adapter in the direction towards an attachment point on the adapter for attaching to a double-stranded polynucleotide.

In one embodiment the polynucleotide-handling enzyme is oriented on the polynucleotide adapter to control the movement of the target polynucleotide with respect to a detector such as a nanopore in a second direction opposite to the first direction. The second direction may for instance be in the trans-to-cis direction. The polynucleotide-handling enzyme may be oriented to process the polynucleotide adapter in the direction away from an attachment point on the adapter for attaching to a double-stranded polynucleotide.

### Kit

Also provided are kits comprising polynucleotide adapters, motor proteins and polynucleotide-handling enzymes. It will be understood that any of the polynucleotide adapters disclosed herein can be applied in the embodiments of the kits discussed herein and above.

In one embodiment, provided is a kit for modifying a target polynucleotide, comprising:

i) a polynucleotide adapter;

ii) a motor protein capable of controlling the movement of a target polynucleotide in a first direction with respect to a nanopore, wherein the active double stranded polynucleotide-unwinding activity of the motor protein is suppressed; and

iii) a polynucleotide-handling enzyme capable of applying a force to move the target polynucleotide in a second direction opposite to the first direction.

In one embodiment, the polynucleotide adapter is a polynucleotide adapter as described in more detailed herein. In one embodiment, the motor protein is a motor protein as described herein. In one embodiment the polynucleotide-handling enzyme is a blocking moiety as described herein.

In one embodiment the motor protein configured to orient on the polynucleotide adapter to control the movement of the target polynucleotide with respect to a nanopore in a first direction. In one embodiment the motor protein is provided in a form adapted to orient on the polynucleotide adapter to control the movement of the target polynucleotide with respect to a detector such as a nanopore in a first direction. The first direction may for instance be in the cis-to-trans direction. The motor protein may thus be provided in a form to orient to process the polynucleotide adapter in the direction towards an attachment point on the adapter for attaching to a double-stranded polynucleotide. For example, the motor protein may be provided on a polynucleotide configured to deliver the motor protein to the adapter in the desired orientation.

In one embodiment the polynucleotide-handling enzyme configured to orient on the polynucleotide adapter to control the movement of the target polynucleotide with respect to a detector such as a nanopore in a second direction opposite to the first direction. In one embodiment the polynucleotide-handling enzyme is provided in a form adapted to orient on the polynucleotide adapter to control the movement of the target polynucleotide with respect to a nanopore in a second direction opposite to the first direction. The second direction may for instance be in the trans-to-cis direction. The polynucleotide-handling enzyme may thus be provided in a form to orient to process the polynucleotide adapter in the direction away from an attachment point on the adapter for attaching to a double-stranded polynucleotide. For example, the polynucleotide-handling enzyme may be provided on a polynucleotide configured to deliver the polynucleotide-handling enzyme to the adapter in the desired orientation.

In one embodiment, provided is a kit comprising:

i) a polynucleotide adapter;

ii) a motor protein capable of controlling the movement of a target polynucleotide in a first direction with respect to a nanopore, wherein the active double stranded polynucleotide-unwinding activity of the motor protein is suppressed; and

iii) a controllable anchoring point (e.g. an AFM tip) for the target polynucleotide capable of applying a force to move the target polynucleotide in a second direction opposite to the first direction.

In one embodiment, the polynucleotide adapter is a polynucleotide adapter as described in more detailed herein. In one embodiment, the motor protein is a motor protein as described herein. In one embodiment the controllable anchoring point is a controllable anchoring point (e.g. an AFM tip) as described herein.

### System

Also provided are systems comprising polynucleotide adapters, motor proteins, polynucleotide-handling enzymes and nanopores. It will be understood that any of the polynucleotide adapters disclosed herein can be applied in the embodiments of the systems discussed herein and above.

In one embodiment provided is a system for characterising a target double-stranded polynucleotide comprising:
a polynucleotide adapter;
a nanopore for characterising the target polynucleotide as the target polynucleotide moves with respect to the nanopore
a motor protein for moving the double-stranded polynucleotide in a first direction relative to the nanopore; and
a polynucleotide-handling enzyme for moving the double-stranded polynucleotide in a second direction relative to the nanopore.

In one embodiment, the polynucleotide adapter is a polynucleotide adapter as described in more detailed herein. In one embodiment, the motor protein is a motor protein as described herein. In one embodiment the polynucleotide-handling enzyme is a polynucleotide-handling enzyme as described herein. In one embodiment the nanopore is a nanopore as described herein. The system may further comprise a membrane; control equipment; etc as defined herein.

In another embodiment provided is a system for characterising a target double-stranded polynucleotide comprising:
a polynucleotide adapter;
a nanopore for characterising the target polynucleotide as the target polynucleotide moves with respect to the nanopore
a motor protein for moving the double-stranded polynucleotide in a first direction relative to the nanopore; and
a controllable anchoring point (e.g. an AFM tip) to which the target polynucleotide may be attached, for moving the double-stranded polynucleotide in a second direction relative to the nanopore.

In one embodiment, the polynucleotide adapter is a polynucleotide adapter as described in more detailed herein. In one embodiment, the motor protein is a motor protein as described herein. In one embodiment the controllable anchoring point is a controllable anchoring point (e.g. an AFM tip) as described herein. In one embodiment the nanopore is a nanopore as described herein. The system may further comprise a membrane; control equipment; etc as defined herein.

It is to be understood that although particular embodiments, specific configurations as well as materials and/or molecules, have been discussed herein for methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1: DNA Sequencing by Fuel-Free Sliding

This example describes a method of controlling the movement of a DNA strand through a nanopore under an applied potential using a motor protein (TraI Cba helicase with the mutations L376C/Q594A/K762C) which has been mutated to prevent ATP hydrolysis.
A Y adapter was prepared as follows:
Top strand (SEQ ID NO: 10), bottom strand (SEQ ID NO: 11) and blocker DNA strand (SEQ ID NO: 12) were annealed, and the annealed DNA was incubated with TraI Cba-L376C/Q594A/K762C at room temperature for 1 hour in buffer (50 mM CAPS pH 10, 100 mM KCl, 1 mM EDTA). 0.1× volume of Tris at pH 7 was added to lower the pH, followed by 0.1× volume of BMOE (bis-maleimidoethane), and incubated at room temperature for 1 hour. The protein bound Y adapter was HPLC purified—the purified protein bound Y adapter will now be referred to as “Y adapter.”
An A-tailed DNA analyte was prepared as follows:
Zymo Mock Community DNA (ZymoBIOMICS) was fragmented with a Covaris g-tube (Covaris) at 3000 rpm, PCR amplified, then treated using NEBNext end repair and NEBNext dA-tailing modules (New England Biolabs (NEB)), to generate 3′ A overhangs.
The Y adapter was ligated to the A-tailed DNA analyte according to Oxford Nanopore's LSK109 protocol (see https://community.nanoporetech.com/protocols/gDNA-sqk-lsk109/v/gde_9063_v109_revt_14 aug2019 for details): Y adapter, A-tailed DNA, T4 DNA ligase (NEB) and LNB (Oxford Nanopore) were combined according to the recipe in the table below, and incubated at room temperature for 10 minutes. The Y adapter, A-tailed DNA, T4 DNA ligase (NEB) and LNB (Oxford Nanopore) mixture was then purified by adding a 0.4× volume of Agencourt AMPure XP (Beckman Coulter) beads and incubating at room temperature for 10 minutes. The beads were washed twice with SFB (Oxford Nanopore) and eluted in 40 μL EB (Oxford Nanopore) for 10 minutes at room temperature (now referred to as “eluted DNA”). 10 uL eluted DNA was added to 65 uL buffer (500 mM KCl, 25 mM HEPES pH 8, 1 mM EDTA, known as “running buffer”), this is hence referred to as “sequencing mix”.

### DNA Ligation Recipe for Example 1

| | | |
|---|---|---|
| Reagent | Volume | Final Concentration |

| A tailed DNA | 6 | uL | 2 | ug |
|---|---|---|---|---|
| Nuclease free water | 49 | uL | | |
| LNB (Oxford Nanopore) | 25 | uL | 1x | |
| T4 DNA ligase (NEB) | 10 | uL | | |
| Y adapter | 10 | uL | 200 | nM |

| | | |
|---|---|---|
| TOTAL | 100 | uL |

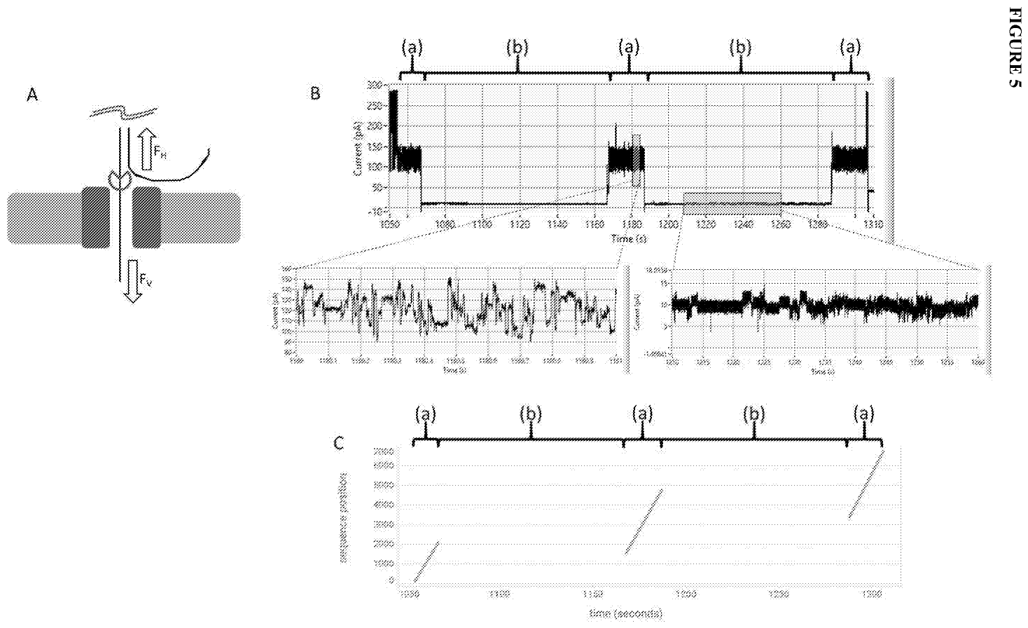

Electrical measurements were acquired from single CsgG nanopores inserted in block co-polymer in buffer (150 mM potassium ferricyanide, 150 mM potassium ferrocyanide, 25 mM potassium phosphate pH 8, known as “mediator buffer”). After achieving a single pore inserted in the block co polymer, then 1 mL mediator buffer containing 400 nM thrombin binding aptamer was flowed through the system to remove any excess CsgG nanopores. 1 mL running buffer was then flowed through the system, followed by 75 uL sequencing mix.
The experiment was controlled using a custom script in MinKNOW software (Oxford Nanopore Technologies). The custom script applies a global voltage of 180 mV, and applies an unblocking voltage of −300 mV to channels which are enter an unproductive state. Raw data is recorded for subsequent analysis.
The raw data (FIG. 4) showed examples of DNA translocating the nanopore under the control of the motor protein. The raw data translocation events were basecalled using a retrained RNN with Guppy software (v3.1.5, Oxford Nanopore), and the basecalls were aligned to the reference genome usingminimap2 (https://github.com/lh3/minimap2, version 2.14-r883). The basecall alignments in conjunction with the translocation times could be used to calculate the average translocation rate in bases per second. In this experiment the average translocation rate across all strands was 106 bases per second.

### Example 2: DNA Re-Reading by Static Global Voltage Cycling

This example describes a method of moving a DNA strand back and forth through a nanopore multiple times under the control of an inactive motor protein (TraI Cba helicase with the mutations L376C/Q594A/K762C) by varying the applied potential. The re-hybridisation of newly separated double stranded DNA generates a force in the opposite direction to that of the applied potential. The DNA can be moved through the nanopore in either direction by changing the applied potential—when the applied potential is stronger than the hybridisation force, the DNA moves into the pore; when the applied potential is weaker than the hybridisation force, the DNA moves out of the pore. By cycling between an applied potential of 180 mV and 20 mV, DNA strands were moved back and forth through a nanopore, allowing the same section of DNA to be sequenced multiple times.
A Y adapter was prepared as in Example 1.
A double stranded DNA analyte was prepared using NEBNext end repair and NEBNext dA-tailing modules (New England Biolabs (NEB)), to generate 3′ A overhangs.
The Y adapter was ligated to the A-tailed DNA analyte according to Oxford Nanopore's LSK109 protocol (https://community.nanoporetech.com/protocols/gDNA-sqk-lsk109/v/gde_9063_v109_revt_14 aug2019): Y adapter, A-tailed DNA, T4 DNA ligase (NEB) and LNB (Oxford Nanopore) were combined according to the recipe in the table below, and incubated at room temperature for 10 minutes. The Y adapter, A-tailed DNA, T4 DNA ligase (NEB) and LNB (Oxford Nanopore) mixture was then purified by adding a 0.4× volume of Agencourt AMPure XP (Beckman Coulter) beads and incubating at room temperature for 10 minutes. The beads were washed twice with SFB (Oxford Nanopore) and eluted in 40 μL EB (Oxford Nanopore) for 10 minutes at room temperature (now referred to as “eluted DNA”). 5 uL eluted DNA was added to 70 uL buffer (500 mM KCl, 25 mM HEPES pH 8, 1 mM EDTA, known as “running buffer”), this is hence referred to as “sequencing mix”.

### DNA Ligation Recipe for Example 2

| | | |
|---|---|---|
| Reagent | Volume | Final Concentration |

| A tailed DNA | 5 | uL | 2 | ug |
|---|---|---|---|---|
| Nuclease free water | 50 | uL | | |
| LNB (Oxford Nanopore) | 25 | uL | 1x | |
| T4 DNA ligase (NEB) | 10 | uL | | |
| Y adapter | 10 | uL | 200 | nM |

| | | |
|---|---|---|
| TOTAL | 100 | uL |

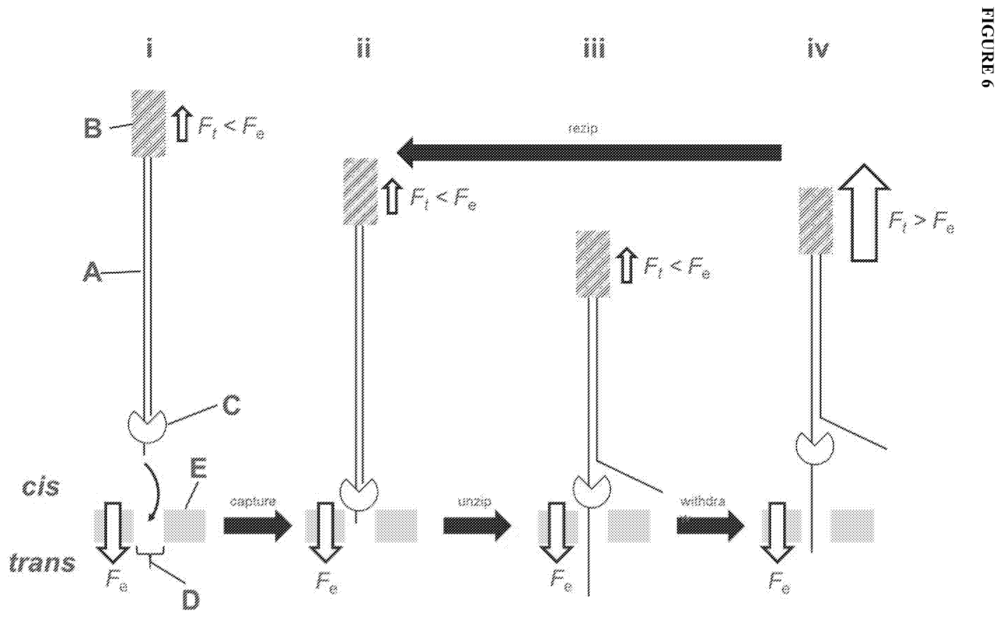

Electrical measurements were acquired from single CsgG nanopores inserted in block co-polymer in buffer (150 mM potassium ferricyanide, 150 mM potassium ferrocyanide, 25 mM potassium phosphate pH 8, known as “mediator buffer”). After achieving a single pore inserted in the block co polymer, then 1 mL mediator buffer containing 400 nM thrombin binding aptamer was flowed through the system to remove any excess CsgG nanopores. 1 mL running buffer was then flowed through the system, followed by 75 uL sequencing mix.
The experiment was controlled using a custom script in MinKNOW software (Oxford Nanopore Technologies). The custom script applies a global voltage of 180 mV for 7 seconds, then 20 mV for 40 seconds. This cycle of voltages was repeated for 12 hours. Raw data was recorded for subsequent analysis.
The raw data (FIG. 5) contained examples of controlled DNA translocation while the applied voltage was set to 180 mV. When the voltage was lowered to 20 mV, and then set to 180 mV again, the recorded current did not return to the open pore current level—this showed that the DNA strand had not exited the pore, and that the same DNA strand was measured in each translocation event. The raw data translocation events were basecalled using a retrained RNN with Guppy software (v3.1.5, Oxford Nanopore), and the basecalls were aligned to the reference genome using minimap2 (https://github.com/lh3/minimap2, version 2.14-r883). The basecall alignments showed that the DNA strand moved into the pore when the applied potential was 180 mV at an average rate of 90 bases per second, and moved out of the pore when the applied potential was 20 mV at an average rate of 9 bases per second.

### Example 3

This example describes how a polynucleotide analyte may be re-read multiple times by using an AFM tip attachment to reset the position of DNA in the pore. The DNA movement is controlled both by using an inactivated motor protein (TraI Cba helicase with the mutations L376C/Q594A/K762C which has been mutated to prevent ATP hydrolysis), and the AFM tip.
A Y adapter is prepared as follows:
Top strand (SEQ ID NO: 10), bottom strand (SEQ ID NO: 11) and blocker DNA strand (SEQ ID NO: 12) are annealed, and the annealed DNA incubated with TraI Cba-L376C/Q594A/K762C at room temperature for 1 hour in buffer (50 mM CAPS pH 10, 100 mM KCl, 1 mM EDTA). 0.1× volume of Tris at pH 7 is added to lower the pH, followed by 0.1× volume of BMOE (bis-maleimidoethane), and incubated at room temperature for 1 hour. The protein bound Y adapter is purified by HPLC. The purified protein bound Y adapter will henceforth be referred to as “Y adapter.”
A biotin attachment adapter is prepared as follows:
Top strand (SEQ ID NO: 13) and bottom strand SEQ ID NO: 14) are annealed together in an equimolar ratio.
An A-tailed DNA analyte is prepared as follows:
Zymo Mock Community DNA (ZymoBIOMICS) is fragmented with a Covaris g-tube (Covaris) at 3000 rpm, PCR amplified, then treated using NEBNext end repair and NEBNext dA-tailing modules (New England Biolabs (NEB)), to generate 3′ A overhangs.
The Y adapter is ligated to the A-tailed DNA analyte according to Oxford Nanopore's LSK109 protocol (see https://community.nanoporetech.com/protocols/gDNA-sqk-lsk109/v/gde_9063_v109_revt_14 aug2019 for details): Y adapter, biotin attachment adapter, A-tailed DNA, T4 DNA ligase (NEB) and LNB (Oxford Nanopore) are combined according to the recipe in the table below, and incubated at room temperature for 10 minutes.

| | | |
|---|---|---|
| Reagent | Volume | Final Concentration |

| A tailed DNA | 5 | uL | 2 | ug |
|---|---|---|---|---|
| Nuclease free water | 50 | uL | | |
| LNB (Oxford Nanopore) | 25 | uL | 1x | |
| T4 DNA ligase (NEB) | 10 | uL | | |
| Y adapter | 5 | uL | 100 | nM |
| Biotin attachment adapter | 5 | uL | 100 | nM |

| | | |
|---|---|---|
| TOTAL | 100 | uL |

The Y adapter, A-tailed DNA, T4 DNA ligase (NEB) and LNB (Oxford Nanopore) mixture are then purified by adding a 0.4× volume of Agencourt AMPure XP (Beckman Coulter) beads and incubating at room temperature for 10 minutes. The beads are washed twice with SFB (Oxford Nanopore) and eluted in 40 μL EB (Oxford Nanopore) for 10 minutes at room temperature (now referred to as “eluted DNA”). 10 uL eluted DNA are added to 65 uL buffer (500 mM KCl, 25 mM HEPES pH 8, 1 mM EDTA, known as “running buffer”) and 50 nM streptavidin. This is hence referred to as “sequencing analyte”.
An AFM tip is biotinylated by silanisation with (3-aminopropyl)triethoxysilane, followed by reaction with NHS-PEG-biotin.
Electrical measurements are acquired using Oxford Nanopore Technologies' MinION flow cells, via single CsgG nanopores inserted in block co-polymer in buffer (150 mM potassium ferricyanide, 150 mM potassium ferrocyanide, 25 mM potassium phosphate pH 8, known as “mediator buffer”). After achieving a single pore per channel inserted in the block co polymer, then 1 mL mediator buffer containing 400 nM thrombin binding aptamer is flowed through the system to remove any excess CsgG nanopores. 1 mL running buffer is then flowed through the system, followed by 75 uL sequencing mix via the SpotON port. The AFM tip is introduced via the SpotON port and incubated for 1 hr to bind DNA library. A sequencing voltage (180 mV) is applied across the membrane in the cis to trans direction. Electrical measurements are taken corresponding to the channels directly below the SpotON port. The AFM tip is lowered to within a few microns of the membrane until DNA is captured by a nanopore, as judged by a blockade event from open pore level. The AFM tip is controlled by an atomic force microscope in constant force mode (˜10 pN), with periodic increases in the force (approximately equal to or above that of the electrical force) to partially reverse the DNA out of the nanopore and so permit reannealing of the double-stranded DNA.

FIG. 6 shows the set-up of the experiment described in this example. FIG. 7 shows the expected current-time traces and inactivated helicase position from this example in relation to the force applied by the AFM tip.

### Description of the Sequence Listing

SEQ ID NO: 1 shows the amino acid sequence of (hexa-histidine tagged) exonuclease I (EcoExo I) from E. coli.
SEQ ID NO: 2 shows the amino acid sequence of the exonuclease III enzyme from E. coli. SEQ ID NO: 3 shows the amino acid sequence of the RecJ enzyme from T. thermophilus (TthRecJ-cd).
SEQ ID NO: 4 shows the amino acid sequence of bacteriophage lambda exonuclease. The sequence is one of three identical subunits that assemble into a trimer. (neb.com/nebecomm/products/productM0262.asp).
SEQ ID NO: 5 shows the amino acid sequence of Phi29 DNA polymerase from Bacillus subtilis phage Phi29.
SEQ ID NO: 6 shows the amino acid sequence of Trwc Cba (Citromicrobium bathyornarinum) helicase.
SEQ ID NO: 7 shows the amino acid sequence of Hel308 Mbu (Methanococcoides burtonii) helicase.
SEQ ID NO: 8 shows the amino acid sequence of the Dda helicase 1993 from Enterobacteria phage T4.
SEQ ID NO: 9 shows the amino acid sequence of TraI Cba (Citromicrobium bathyornarinum) helicase L376C/Q594A/K762C.
SEQ ID NO: 10 shows the nucleotide of the top strand of the Y adapter used in Example 1.
SEQ ID NO: 11 shows the nucleotide of the bottom strand of the Y adapter used in Example 1.
SEQ ID NO: 12 shows the nucleotide of the blocker strand of the Y adapter used in Example 1.
SEQ ID NO: 13 shows the polynucleotide sequence of the top strand of the biotin attachment adapter used in Example 3. (/5Phos/ is 5′ phosphate)
SEQ ID NO: 14 shows the polynucleotide sequence of the top strand of the biotin attachment adapter used in Example 3. (/5Phos/ is 5′ phosphate; /3Biotin-TEG/ is 3′ biotin, via TEG linker)

SEQUENCE LISTING
exonuclease 1 from E. coli
SEQ ID NO: 1
MMNDGKQQSTFLFHDYETFGTHPALDRPAQFAAIR
TDSEFNVIGEPEVFYCKPADDYLPQPGAVLITGIT
PQEARAKGENEAAFAARIHSLFTVPKTCILGYNNV
RFDDEVTRNIFYRNFYDPYAWSWQHDNSRWDLLDV
MRACYALRPEGINWPENDDGLPSFRLEHLTKANGI
EHSNAHDAMADVYATIAMAKLVKTRQPRLFDYLFT
HRNKHKLMALIDVPQMKPLVHVSGMFGAWRGNTSW
VAPLAWHPENRNAVIMVDLAGDISPLLELDSDTLR
ERLYTAKTDLGDNAAVPVKLVHINKCPVLAQANTL
RPEDADRLGINRQHCLDNLKILRENPQVREKVVAI
FAEAEPFTPSDNVDAQLYNGFFSDADRAAMKIVLE
TEPRNLPALDITFVDKRIEKLLFNYRARNFPGTLD
YAEQQRWLEHRRQVFTPEFLQGYADELQMLVQQYA
DDKEKVALLKALWQYAEEIVSGSGHHHHHH
exonuclease III enzyme from E. coli
SEQ ID NO: 2
MKFVSFNINGLRARPHQLEAIVEKHQPDVIGLQET
KVHDDMFPLEEVAKLGYNVFYHGQKGHYGVALLTK
ETPIAVRRGFPGDDEEAQRRIIMAEIPSLLGNVTV
INGYFPQGESRDHPIKFPAKAQFYQNLQNYLETEL
KRDNPVLIMGDMNISPTDLDIGIGEENRKRWLRTG
KCSFLPEEREWMDRLMSWGLVDTFRHANPQTADRF
SWFDYRSKGFDDNRGLRIDLLLASQPLAECCVETG
IDYEIRSMEKPSDHAPVWATFRR
RecJ enzyme from T. thermophilus
SEQ ID NO: 3
MFRRKEDLDPPLALLPLKGLREAAALLEEALRQGK
RIRVHGDYDADGLTGTAILVRGLAALGADVHPFIP
HRLEEGYGVLMERVPEHLEASDLFLTVDCGITNHA
ELRELLENGVEVIVTDHHTPGKTPPPGLVVHPALT
PDLKEKPTGAGVAFLLLWALHERLGLPPPLEYADL
AAVGTIADVAPLWGWNRALVKEGLARIPASSWVGL
RLLAEAVGYTGKAVEVAFRIAPRINAASRLGEAEK
ALRLLLTDDAAEAQALVGELHRLNARRQTLEEAML
RKLLPQADPEAKAIVLLDPEGHPGVMGIVASRILE
ATLRPVFLVAQGKGTVRSLAPISAVEALRSAEDLL
LRYGGHKEAAGFAMDEALFPAFKARVEAYAARFPD
PVREVALLDLLPEPGLLPQVFRELALLEPYGEGNP
EPLFL
bacteriophage lambda exonuclease
SEQ ID NO: 4
MTPDIILQRTGIDVRAVEQGDDAWHKLRLGVITAS
EVHNVIAKPRSGKKWPDMKMSYFHTLLAEVCTGVA
PEVNAKALAWGKQYENDARTLFEFTSGVNVTESPI
IYRDESMRTACSPDGLCSDGNGLELKCPFTSRDFM
KFRLGGFEAIKSAYMAQVQYSMWVTRKNAWYFANY
DPRMKREGLHYVVIERDEKYMASFDEIVPEFIEKM
DEALAEIGFVFGEQWR
Phi29 DNA polymerase
SEQ ID NO: 5
MKHMPRKMYSCAFETTTKVEDCRVWAYGYMNIEDH
SEYKIGNSLDEFMAWVLKVQADLYFHNLKFDGAFI
INWLERNGFKWSADGLPNTYNTIISRMGQWYMIDI
CLGYKGKRKIHTVIYDSLKKLPFPVKKIAKDFKLT
VLKGDIDYHKERPVGYKITPEEYAYIKNDIQIIAE
ALLIQFKQGLDRMTAGSDSLKGFKDIITTKKFKKV
FPTLSLGLDKEVRYAYRGGFTWLNDRFKEKEIGEG
MVFDVNSLYPAQMYSRLLPYGEPIVFEGKYVWDED
YPLHIQHIRCEFELKEGYIPTIQIKRSRFYKGNEY
LKSSGGEIADLWLSNVDLELMKEHYDLYNVEYISG
LKFKATTGLFKDFIDKWTYIKTTSEGAIKQLAKLM
LNSLYGKFASNPDVTGKVPYLKENGALGFRLGEEE
TKDPVYTPMGVFITAWARYTTITAAQACYDRIIYC
DTDSIHLTGTEIPDVIKDIVDPKKLGYWAHESTFK
RAKYLRQKTYIQDIYMKEVDGKLVEGSPDDYTDIK
FSVKCAGMTDKIKKEVTFENFKVGFSRKMKPKPVQ
VPGGWLVDDTFTIKSGGSAWSHPQFEKGGGSGGGS
GGSAWSHPQFEK
Trwc Cba helicase
SEQ ID NO: 6
MLSVANVRSPSAAASYFASDNYYASADADRSGQWI
GDGAKRLGLEGKVEARAFDALLRGELPDGSSVGNP
GQAHRPGTDLTFSVPKSWSLLALVGKDERIIAAYR
EAVVEALHWAEKNAAETRVVEKGMVVTQATGNLAI
GLFQHDTNRNQEPNLHFHAVIANVTQGKDGKWRTL
KNDRLWQLNTTLNSIAMARFRVAVEKLGYEPGPVL
KHGNFEARGISREQVMAFSTRRKEVLEARRGPGLD
AGRIAALDTRASKEGIEDRATLSKQWSEAAQSIGL
DLKPLVDRARTKALGQGMEATRIGSLVERGRAWLS
RFAAHVRGDPADPLVPPSVLKQDRQTIAAAQAVAS
AVRHLSQREAAFERTALYKAALDFGLPTTIADVEK
RTRALVRSGDLIAGKGEHKGWLASRDAVVTEQRIL
SEVAAGKGDSSPAITPQKAAASVQAAALTGQGFRL
NEGQLAAARLILISKDRTIAVQGIAGAGKSSVLKP
VAEVLRDEGHPVIGLAIQNTLVQMLERDTGIGSQT
LARFLGGWNKLLDDPGNVALRAEAQASLKDHVLVL
DEASMVSNEDKEKLVRLANLAGVHRLVLIGDRKQL
GAVDAGKPFALLQRAGIARAEMATNLRARDPVVRE
AQAAAQAGDVRKALRHLKSHTVEARGDGAQVAAET
WLALDKETRARTSIYASGRAIRSAVNAAVQQGLLA
SREIGPAKMKLEVLDRVNTTREELRHLPAYRAGRV
LEVSRKQQALGLFIGEYRVIGQDRKGKLVEVEDKR
GKRFRFDPARIRAGKGDDNLTLLEPRKLEIHEGDR
IRWTRNDHRRGLFNADQARVVEIANGKVTFETSKG
DLVELKKDDPMLKRIDLAYALNVHMAQGLTSDRGI
AVMDSRERNLSNQKTFLVTVTRLRDHLTLVVDSAD
KLGAAVARNKGEKASAIEVTGSVKPTATKGSGVDQ
PKSVEANKAEKELTRSKSKTLDFGI
Hel308 Mbu helicase
SEQ ID NO: 7
MMIRELDIPRDIIGFYEDSGIKELYPPQAEAIEMG
LLEKKNLLAAIPTASGKTLLAELAMIKAIREGGKA
LYIVPLRALASEKFERFKELAPFGIKVGISTGDLD
SRADWLGVNDIIVATSEKTDSLLRNGTSWMDEITT
VVVDEIHLLDSKNRGPTLEVTITKLMRLNPDVQVV
ALSATVGNAREMADWLGAALVLSEWRPTDLHEGVL
FGDAINFPGSQKKIDRLEKDDAVNLVLDTIKAEGQ
CLVFESSRRNCAGFAKTASSKVAKILDNDIMIKLA
GIAEEVESTGETDTAIVLANCIRKGVAFHHAGLNS
NHRKLVENGFRQNLIKVISSTPTLAAGLNLPARRV
IIRSYRRFDSNFGMQPIPVLEYKQMAGRAGRPHLD
PYGESVLLAKTYDEFAQLMENYVEADAEDIWSKLG
TENALRTHVLSTIVNGFASTRQELFDFFGATFFAY
QQDKWMLEEVINDCLEFLIDKAMVSETEDIEDASK
LFLRGTRLGSLVSMLYIDPLSGSKIVDGFKDIGKS
TGGNMGSLEDDKGDDITVTDMTLLHLVCSTPDMRQ
LYLRNTDYTIVNEYIVAHSDEFHEIPDKLKETDYE
WFMGEVKTAMLLEEWVTEVSAEDITRHFNVGEGDI
HALADTSEWLMHAAAKLAELLGVEYSSHAYSLEKR
IRYGSGLDLMELVGIRGVGRVRARKLYNAGFVSVA
KLKGADISVLSKLVGPKVAYNILSGIGVRVNDKHF
NSAPISSNTLDTLLDKNQKTFNDFQ
Dda helicase
SEQ ID NO: 8
MTFDDLTEGQKNAFNIVMKAIKEKKHHVTINGPAG
TGKTTLTKFIIEALISTGETGIILAAPTHAAKKIL
SKLSGKEASTIHSILKINPVTYEENVLFEQKEVPD
LAKCRVLICDEVSMYDRKLFKILLSTIPPWCTIIG
IGDNKQIRPVDPGENTAYISPFFTHKDFYQCELTE
VKRSNAPIIDVATDVRNGKWIYDKVVDGHGVRGFT
GDTALRDFMVNYFSIVKSLDDLFENRVMAFTNKSV
DKLNSIIRKKIFETDKDFIVGEIIVMQEPLFKTYK
IDGKPVSEIIFNNGQLVRIIEAEYTSTFVKARGVP
GEYLIRHWDLTVETYGDDEYYREKIKIISSDEELY
KFNLFLGKTAETYKNWNKGGKAPWSDFWDAKSQFS
KVKALPASTFHKAQGMSVDRAFIYTPCIHYADVEL
AQQLLYVGVTRGRYDVFYV
TraI Cba L376C/Q594A/K762C
SEQ ID NO: 9
MLSVANVRSPSAAASYFASDNYYASADADRSGQWI
GDGAKRLGLEGKVEARAFDALLRGELPDGSSVGNP
GQAHRPGTDLTFSVPKSWSLLALVGKDERIIAAYR
EAVVEALHWAEKNAAETRVVEKGMVVTQATGNLAI
GLFQHDTNRNQEPNLHFHAVIANVTQGKDGKWRTL
KNDRLWQLNTTLNSIAMARFRVAVEKLGYEPGPVL
KHGNFEARGISREQVMAFSTRRKEVLEARRGPGLD
AGRIAALDTRASKEGIEDRATLSKQWSEAAQSIGL
DLKPLVDRARTKALGQGMEATRIGSLVERGRAWLS
RFAAHVRGDPADPLVPPSVLKQDRQTIAAAQAVAS
AVRHLSQREAAFERTALYKAALDFGCPTTIADVEK
RTRALVRSGDLIAGKGEHKGWLASRDAVVTEQRIL
SEVAAGKGDSSPAITPQKAAASVQAAALTGQGFRL
NEGQLAAARLILISKDRTIAVQGIAGAGKSSVLKP
VAEVLRDEGHPVIGLAIQNTLVQMLERDTGIGSQT
LARFLGGWNKLLDDPGNVALRAEAQASLKDHVLVL
DEASMVSNEDKEKLVRLANLAGVHRLVLIGDRKAL
GAVDAGKPFALLQRAGIARAEMATNLRARDPVVRE
AQAAAQAGDVRKALRHLKSHTVEARGDGAQVAAET
WLALDKETRARTSIYASGRAIRSAVNAAVQQGLLA
SREIGPAKMKLEVLDRVNTTREELRHLPAYRAGRV
LEVSRKQQALGLFIGEYRVIGQDRKGCLVEVEDKR
GKRFRFDPARIRAGKGDDNLTLLEPRKLEIHEGDR
IRWTRNDHRRGLFNADQARVVEIANGKVTFETSKG
DLVELKKDDPMLKRIDLAYALNVHMAQGLTSDRGI
AVMDSRERNLSNQKTFLVTVTRLRDHLTLVVDSAD
KLGAAVARNKGEKASAIEVTGSVKPTATKGSGVDQ
PKSVEANKAEKELTRSKSKTLDFGI
Top strand
SEQ ID NO: 10
/5SpC3//iSpC3//iSpC3//iSpC3//iSpC3//
iSpC3//iSpC3//iSpC3//iSpC3//iSpC3//
iSpC3//iSpC3//iSpC3//iSpC3//iSpC3//
iSpC3//iSpC3//iSpC3//iSpC3//iSpC3//
iSpC3//iSpC3//iSpC3//iSpC3//iSpC3//
iSpC3//iSpC3//iSpC3//iSpC3//iSpC3/
GGTTGTTTCTGTTGGTGCTGATATTGCTTTTTTTT
TTTTTTTTTTGGACACCTCGTCGCTAGTCGCT
Bottom strand
SEQ ID NO: 11
GCGACTAGCGACGAGGTGTCCTTTGAGGCGAGCGGT
CAA
blocker strand
SEQ ID NO: 12
CCAACAGAAACAACC
SEQ ID NO: 13
/5Phos/GGACACCTCGTCGCTAGTCGCT
SEQ ID NO: 14
/5Phos/GCGACTAGCGACGAGGTGTCCT/
3Biotin-TEG/

1. A method of moving a double-stranded polynucleotide with respect to a nanopore, comprising:
a) contacting the polynucleotide with a motor protein and a nanopore;
b) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;
c) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises; and
d) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore;
wherein the active double stranded polynucleotide-unwinding activity of the motor protein is suppressed.

2. A method according to claim 1, wherein the first portion of the double-stranded polynucleotide is the same as the second portion of the double-stranded polynucleotide.

3. A method according to claim 1, wherein the first portion of the double-stranded polynucleotide partially overlaps with the second portion of the double-stranded polynucleotide.

4. A method according to any one of the preceding claims, further comprising:
e) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises.

5. A method according to claim 4, wherein steps (d) and (e) are repeated multiple times.

6. A method according to claim 5, wherein, at each repeat, the second portion of the double-stranded polynucleotide partially overlaps with the second portion of the double-stranded polynucleotide of the preceding repeat.

7. A method according to any one of the preceding claims, wherein allowing the double-stranded polynucleotide to move in the first direction with respect to the nanopore comprises applying a first force to the double-stranded polynucleotide.

8. A method according to claim 7, wherein the first force exceeds the rehybridization force of the polynucleotide.

9. A method according to any one of the preceding claims, wherein allowing the double-stranded polynucleotide to move in the second direction with respect to the nanopore comprises applying a second force to the double-stranded polynucleotide.

10. A method according to claim 9, wherein the second force is applied in the same direction relative to the nanopore as the first force, and the second force is exceeded by the rehybridization force of the polynucleotide.

11. A method according to any one of claims 7 to 10, wherein the first force and/or the second force comprises a voltage potential.

12. A method according to claim 11, wherein the first force comprises a voltage potential and the second force comprises a voltage potential, and the first force is greater than the second force.

13. A method according to any one of claims 9 to 12, wherein the second force is applied in the opposite direction relative to the nanopore as the first force.

14. A method according to any one of claims 9 to 13, wherein the second force comprises a force applied by a polynucleotide-handling enzyme which moves the polynucleotide in the second direction relative to the nanopore.

15. A method according to any one of claims 7 to 14, wherein the first force comprises a voltage potential and the second force comprises (i) a voltage potential applied in the same direction relative to the nanopore as the first force; and (ii) a force applied by a polynucleotide-handling enzyme which moves the polynucleotide in the opposite direction relative to the nanopore as the first force;
and wherein the component of the second force applied by the polynucleotide-handling enzyme exceeds the component of the second force applied by the voltage potential.

16. A method according to claim 14 or 15, wherein the polynucleotide-handling enzyme is a helicase or a variant thereof; preferably wherein the polynucleotide-handling enzyme comprises the sequence of SEQ ID NO: 7 or a variant thereof or the sequence of SEQ ID NO: 8 or a variant thereof.

17. A method according to any one of the preceding claims, wherein the movement of the polynucleotide in the first direction is faster than the movement of the polynucleotide in the second direction.

18. A method according to any one of the preceding claims, wherein the active double stranded polynucleotide-unwinding activity of the motor protein is suppressed by omitting fuel for the motor protein from the reaction medium.

19. A method according to any one of the preceding claims, wherein the motor protein is a variant in which NTP binding and/or hydrolysis is abolished or suppressed.

20. A method according to any one of the preceding claims, wherein the motor protein is a variant in which DNA-processing activity is abolished or suppressed.

21. A method according to any one of the preceding claims, wherein the motor protein is a helicase variant in which the pin domain has been removed or reduced.

22. A method according to claim any one of the preceding claims, wherein the motor protein is a helicase or a variant thereof; preferably wherein the motor protein comprises the sequence of SEQ ID NO: 6 or a variant thereof.

23. A method of characterising a double-stranded polynucleotide analyte, comprising carrying out a method according to any one of claims 1 to 22; wherein one or more of steps (b), (c), (d) and (e) if present comprise taking one or more measurements as the double stranded polynucleotide moves with respect to the nanopore, wherein the one or more measurements are indicative of one or more characteristics of the polynucleotide, and thereby characterising the polynucleotide as it moves with respect to the nanopore

24. A method of characterising a target double-stranded polynucleotide analyte, comprising:
a) contacting the polynucleotide with a motor protein and a nanopore;
b1) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;
b2) taking one or more or more measurements indicative of one or more characteristics of the target polynucleotide as the double stranded polynucleotide moves in the first direction with respect to the nanopore;
c1) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises;
c2) optionally taking one or more or more measurements indicative of one or more characteristics of the target polynucleotide as the double stranded polynucleotide moves in the second direction with respect to the nanopore;
d1) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore; and
d2) taking one or more or more measurements indicative of one or more characteristics of the target polynucleotide as the double stranded polynucleotide moves in the first direction with respect to the nanopore;
wherein the active double stranded polynucleotide-unwinding activity of the motor protein is suppressed.

25. A method according to claim 24, further comprising:
e1) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises; and
e2) optionally taking one or more or more measurements indicative of one or more characteristics of the target polynucleotide as the double stranded polynucleotide moves in the second direction with respect to the nanopore.

26. A method according to claim 24 or claim 25, wherein:
the first and/or second portions are as defined in any one of claim 2, 3 or 6;
the movement of the polynucleotide is as defined in any one of claim 4, 5, 7-15 or 17;
the motor protein is as defined in any one of claims 18 to 22
the polynucleotide-handling enzyme if present is as defined in claim 16.

27. A method according to any one of claims 23 to 26, wherein the one or more measurements are one or more current measurements and/or one or more optical measurements.

28. A method of encoding data on a double-stranded polynucleotide, comprising carrying out a method according to any one of claims 1 to 22; wherein one or more of steps (b), (c), (d) and (e) if present comprise modifying the portion of the polynucleotide in the vicinity of the nanopore as the polynucleotide moves with respect to the nanopore.

29. A method of encoding data on a double-stranded polynucleotide, comprising:
a) contacting the polynucleotide with a motor protein and a nanopore;
b1) allowing the double-stranded polynucleotide to move in a first direction with respect to the nanopore under conditions such that (i) a first portion of the double-stranded polynucleotide dehybridises and (ii) the motor protein controls the movement of one strand of the first portion of the double-stranded polynucleotide in the first direction with respect to the nanopore;
b2) modifying the portion of the polynucleotide in the vicinity of the nanopore as the double stranded polynucleotide moves in the first direction with respect to the nanopore;
c1) allowing the double-stranded polynucleotide to move in a second direction with respect to the nanopore under conditions such that (i) the strand of the first portion of the double stranded polynucleotide moves in the second direction with respect to the nanopore and (ii) at least part of the first portion of the polynucleotide rehybridises;
c2) optionally modifying the portion of the polynucleotide in the vicinity of the nanopore as the double stranded polynucleotide moves in the second direction with respect to the nanopore;
d1) allowing the double-stranded polynucleotide to move in the first direction with respect to the nanopore under conditions such that (i) a second portion of the double-stranded polynucleotide dehybridises and (ii) the motor protein controls the movement of one strand of the second portion of the double-stranded polynucleotide in the first direction with respect to the nanopore; and
d2) modifying the portion of the polynucleotide in the vicinity of the nanopore as the double stranded polynucleotide moves in the first direction with respect to the nanopore;
wherein the active double stranded polynucleotide-unwinding activity of the motor protein is suppressed.

30. A method according to claim 29, further comprising:
e1) allowing the double-stranded polynucleotide to move in the second direction with respect to the nanopore under conditions such that (i) the strand of the second portion of the double stranded polynucleotide moves in the second direction with respect to the nanopore and (ii) at least part of the second portion of the polynucleotide rehybridises; and
e2) optionally modifying the portion of the polynucleotide in the vicinity of the nanopore as the double stranded polynucleotide moves in the second direction with respect to the nanopore.

31. A method according to claim 29 or claim 30, wherein:
the first and/or second portions are as defined in any one of claim 2, 3 or 6;
the movement of the polynucleotide is as defined in any one of claim 4, 5, 7-15 or 17;
the motor protein is as defined in any one of claims 18 to 22
the polynucleotide-handling enzyme if present is as defined in claim 16.

32. A method according to any one of claims 28 to 31, wherein modifying the polynucleotide comprises subjecting the portion of the polynucleotide in the vicinity of the nanopore to reaction conditions comprising (i) the presence, absence or concentration of one or more chemical reagent(s); (ii) the engagement of an enzyme with the polynucleotide strand under conditions that the enzyme modifies the nucleotides within the polynucleotide strand; (iii) the presence or absence of electromagnetic radiation; and/or (iv) the presence or absence of applied heat.

33. A polynucleotide adapter having a motor protein and a polynucleotide-handling enzyme bound thereto, wherein the motor protein is capable of controlling the movement of the target polynucleotide with respect to a nanopore in a first direction; the polynucleotide-handling enzyme is capable of applying a force to move the target polynucleotide with respect to the nanopore in a second direction opposite to the first direction; and wherein the active double stranded polynucleotide-unwinding activity of the motor protein is suppressed.

34. A kit for modifying a polynucleotide, comprising:
i) a polynucleotide adapter;
ii) a motor protein capable of controlling the movement of a target polynucleotide in a first direction with respect to a nanopore, wherein the active double stranded polynucleotide-unwinding activity of the motor protein is suppressed; and
iii) a polynucleotide-handling enzyme capable of applying a force to move the target polynucleotide in a second direction opposite to the first direction.

35. A polynucleotide adapter or kit according to claim 33 or 34, wherein the motor protein is as defined in any one of claims 18 to 22 and the polynucleotide-handling enzyme is as defined in claim 16.


SEQUENCE LISTING (14 sequences)

SEQ ID NO: 1 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 2 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 3 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 4 - protein, 226 aa, Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 5 - protein, 608 aa, Bacillus subtilis
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 6 - protein, 970 aa, Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 7 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 8 - protein, 439 aa, Artificial Sequence
  Dda helicase
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9 - protein, 970 aa, Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGCPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKALGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGCLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 10 - DNA, 67 bp, Artificial Sequence
  Top strand
  misc_feature (1)..(1): 30iSpC3
ggttgtttctgttggtgctgatattgcttttttttttttttttttggacacctcgtcgctagtcgct

SEQ ID NO: 11 - DNA, 39 bp, Artificial Sequence
  Bottom strand
gcgactagcgacgaggtgtcctttgaggcgagcggtcaa

SEQ ID NO: 12 - DNA, 15 bp, Artificial Sequence
  Blocker strand
ccaacagaaacaacc

SEQ ID NO: 13 - DNA, 22 bp, Artificial Sequence
  Top strand
  misc_feature (1)..(1): 5′ phosphate
ggacacctcgtcgctagtcgct

SEQ ID NO: 14 - DNA, 22 bp, Artificial Sequence
  Bottom strand
  misc_feature (1)..(1): 5′ phosphate
  misc_feature (22)..(22): 3′ biotin via TEG linker
gcgactagcgacgaggtgtcct